(19) United States
(12) Patent Application Publication
(10) Pub. No.: US 2012/0253168 A1
(43) Pub. Date: Oct. 4, 2012

(54) Method and apparatus for producing and detecting non-local effects of substances

(76) Inventors: Huping Hu, Stony Brook, NY (US); Maoxin Wu, Stony Brook, NY (US)

(21) Appl. No.: 13/492,830

(22) Filed: Jun. 9, 2012

Related U.S. Application Data
(63) Continuation-in-part of application No. 11/670,996, filed on Feb. 4, 2007.
(60) Provisional application No. 60/767,009, filed on Feb. 27, 2006.

(51) Int. Cl. A61N 2/04; A61B 5/05; A61B 5/021; A61B 5/0476; A61N 2/00; A61B 5/024

(52) U.S. Cl. 600/409; 600/9; 600/14; 600/508; 600/485; 600/544

(57) ABSTRACT

A method and apparatus are disclosed which produce and detect quantum entanglement and non-local effects of substances on responsive targets such as biological systems. In one embodiment, the method includes the steps of providing two parts of a quantum-entangled medium, applying one part to a biological system such as a human, contacting the other part with a desired substance such as a medication or substance encoded with a message, and detecting change of a biological parameter with a detecting device, whereby a non-local effect of the substance on the said biological system is produced and detected for a beneficial purpose. Also described are a number of implementations.

### CROSS-REFERENCE TO RELATED APPLICATIONS

This application is a continuation-in-part application of U.S. patent application Ser. No. 11/670,996 filed on Feb. 4, 2007, which claims priority from U.S. provisional application Ser. No. 60/767,009 filed on Feb. 27, 2006, which applications are fully incorporated herein by reference.

### FIELD OF THE INVENTION

The invention herein relates to method of producing quantum entanglement, non-local effects of substances through quantum entanglement on responsive targets such as biological and chemical systems, to apparatus for such productions, and to method of using the non-local effects for beneficial purposes.

The invention herein is further related to method of objectively and quantitatively detecting and measuring such non-local effect in biological systems, and to apparatus for such detection and measurement.

### BACKGROUND OF THE INVENTION

Many experiments have shown that quantum entanglement is physically real (see Aspect, A., Dalibard, J., & Roger, G. Experimental test of Bell's inequalities using time-varying analyzers. Phys. Rev. Lett. 49, 1804-1807 (1982)). Indeed, it is ubiquitous in the microscopic world and manifests itself macroscopically under some circumstances (see Ghosh, S., Rosenbaum, T. F., Aeppli, G. & Coppersmith, S. N. Entangled quantum state of magnetic dipoles. Nature 425, 48-51 (2003)). Further, photons are intrinsically quantum objects and natural long-distance carriers of information in both classical and quantum communications (Julsgaard, B., Sherson, J., Cirac, J. I., Fiurasek, J. & Polzik, E. S. Experimental demonstration of quantum memory for light. Nature 432, 482-485 (2004)). Indeed, quantum spins of photons and electrons have now been successfully entangled in various ways for purposes of quantum computation (see Matsukevich, D. N. & Kuzmich, A. Quantum state transfer between matter and light. Science 306, 663-666 (2004)).

However, the essence and implications of quantum entanglement are still hotly debated and largely unknown. For example, it is commonly believed that quantum entanglement alone cannot be used to transmit binary or classical information. Further, despite of the fact that all interactions in biological systems at molecular and sub-molecular levels are quantum interactions in nature, it is commonly believed that quantum effects do not play any roles in biological functions such as perception due to quantum decoherence (see Tegmark, M. The importance of quantum decoherence in brain processes. Phys. Rev., 61E: 4194 (2000)).

My invention and discovery were made against such background. No process has previously been known which can produce non-local effects of substances through quantum entanglement on responsive targets such as biological or chemical systems, so that beneficial effects of the said substances can be delivered through quantum-entangling media such as photons of various sources.

### SUMMARY OF THE INVENTION

I have now invented apparatus and method which produce quantum entanglement, and non-local effects of substances through quantum entanglement on responsive targets such as biological and chemical systems.

We have further invented method and apparatus which detect and measure such non-local effects objectively and quantitatively in biological systems.

The subject invention is originated from my recent research on brain functions and nature of quantum entanglement. I have theorized with my collaborator that nuclear and/or electronic spins inside brain play important roles in certain aspects of brain functions such as perception (Hu, H. P., & Wu, M. X. Spin-mediated consciousness theory. Medical Hypotheses 63, 633-646 (2004); also see arXiv e-print quant-ph/0208068 (2002)). I have thought that one might be able to test this theory by first attempting to entangle these spins with those of a substance such as a general anesthetic through interactions with photons and then observing the resulting brain effects such attempt may produce. I have further thought that the suggested experiment would be feasible if quantum entanglement implies genuine interconnectedness and inseparableness of once interacting quantum entities and is able to influence biological and/or chemical processes. Indeed, instead of armchair debate on how the suggested experiments might not work, I just went ahead and carried out the experiments with the assistance of my collaborator.

The subject invention is therefore based on my realizations that (1) quantum entanglement means genuine interconnectedness and inseparableness of once interacting quantum entities and can be directly sensed and utilized by the entangled quantum entities; (2) it can persist in biological, chemical and other systems at room and higher temperatures despite of quantum decoherence; and (3) it can influence chemical and biochemical reactions, other physical processes and micro- and macroscopic properties of all forms of matters. Therefore, it can be harnessed and developed into useful technologies to serve the mankind in many areas such as health, medicine and even recreation besides the already emerging fields of quantum computation.

For example, using the apparatus and method developed in this invention I have discovered that applying magnetic pulses to a biological system such as the human brain when a substance such as a general anesthetic was placed in between caused the brain to feel the effect of said anesthetic for several hours after the treatment as if the test subject had actually inhaled the same.

For another example, using the apparatus and method developed in this invention I have further discovered that drinking water exposed to magnetic pulses, laser light, microwave or even flashlight when a substance such as a general anesthetic was placed in between also caused the brain to feel the effect of said anesthetic in various degrees as if the test subject had actually inhaled the same.

Further, I have verified as detailed below that said biological effect was the consequence of quantum entanglement between quantum entities inside the biological system such as the human brain and those of the substance under study induced by the photons of the magnetic pulses, laser light, microwave or flashlight.

For yet another example, using the objective and quantitative detecting and measuring apparatus and method developed in this invention, we have further discovered that after consumption by a voluntary human subject of one part of water quantum-entangled with a second part of water as disclosed in this invention, the subject's heart rate was increased by adding a heart stimulant to the second part of water.

Key to the present invention is a quantum-entanglement apparatus which includes a quantum-entanglement generating source, said source emitting a plurality of quantum-entangling members such as photons when said source operates; and a substance disposed adjacent to said source, said substance being responsive to said members; such that when said source emits said members which pass through said substance, said members quantum-entangle with said substance.

Key to the objective and quantitative detection and measurement in biological systems in the present invention is a high-sensitivity and/or high-precision apparatus for detecting and measuring a physiological and/or biological parameter.

In one broad embodiment, the invention provides an apparatus that directly produces non-local effects of various substances such as medications on responsive targets such as biological or chemical systems through quantum-entangling members such as photons.

In another broad embodiment, the invention provides an apparatus that produces media such as water which is quantum-entangled with various substances such as medications through quantum-entangling members such as photons, the media so produced being able to induce non-local effects of said substances on various responsive targets such as biological or chemical systems when said targets are treated with said media.

In yet another broad embodiment, the invention provides an apparatus that produces two or more quantum-entangled media through quantum-entangling members such photons.

In yet another broad embodiment, the invention provides a method for directly producing non-local effects of various substances such as medications on various responsive targets such as biological or chemical systems.

In yet another broad embodiment, the invention provides a method for producing a medium such as water which is quantum-entangled with a substance such as a medication.

In yet another broad embodiment, the invention provides a method for producing non-local effects of various substances such as medications on various responsive targets such as biological or chemical systems through physically treating the said responsive targets with said media such as water which are quantum-entangled with said substances such as medications.

In yet another broad embodiment, the invention provides a method for producing two or more quantum-entangled media through applied or naturally occurring photons or other means.

In yet another broad embodiment, the invention provides a method for remotely producing non-local effects of various substances such as medications on various responsive targets such as biological, chemical and other systems through two parts of a quantum-entangled medium with one part being applied to said responsive targets and another part being entangled with the said substances such as medications through quantum-entangling members such as photons at a remote location of arbitrary distance.

In yet another broad embodiment, the invention provides a method for communicating between two remote locations through two parts of a quantum-entangled medium with one part being applied to a responsive target such as a particular biological, chemical or other system at one location and a second part being subsequently entangled with a particular substance representing a particular message through quantum-entangling members such as photons at a remote location of arbitrary distance.

In yet another broad embodiment, the invention provides a method for quantum-entangling two responsive targets such as two biological systems for beneficial purposes through two parts of a quantum-entangled medium with one part being physically applied to one biological system and a second part being physically applied to a second system.

In yet another broad embodiment, the invention provides a method for directly quantum-entangling two responsive targets such as two biological systems for beneficial purposes through quantum-entangling members such as photons.

In yet another broad embodiment, the invention provides a method and apparatus for objectively and quantitatively detecting and measuring non-local effects in biological systems such as a human.

One benefit of the present invention is that a substance such as a medication can be repeatedly used to obtain a beneficial effect on a biological system without the said biological system physically consuming the said substance. A second benefit of the present invention is that the beneficial effect of a substance such as a medication can be, in one broad embodiment, delivered to a biological system such as a patient from a remote location of arbitrary distance. A third benefit of the present invention is that two parts of a quantum-entangled medium with one part being physically at one location and a second part being physically at another location of arbitrary distance can be, in one broad embodiment, used to transmit an encoded message.

My invention may be more completely understood by reference to the following detailed description considered in connection with the accompanying drawings. However, it should be understood that the drawings are designed for purposes of illustration only and not as a definition of the limits of the invention.

### BRIEF DESCRIPTION OF THE DRAWINGS

FIG. 1A is a schematic view of an apparatus according to one embodiment directly producing non-local effect of a substance on a biological system through quantum entanglement.

FIG. 1B is a schematic view of a method according to another embodiment directly producing non-local effect of a substance on a biological system through quantum entanglement induced by photons emitted by the said biological system.

FIG. 2A is a schematic view of the apparatus illustrated in FIG. 1A producing quantum entanglement between a substance and a medium such as water, and the medium subsequently producing non-local effect of the said substance on a biological system after it is consumed by the biological system.

FIG. 2B is a schematic view of another configuration of the apparatus illustrated in FIG. 2A producing quantum entanglement between a substance and a medium such as water, and the medium subsequently producing non-local effect of the said substance on a biological system after it is consumed by the biological system.

FIG. 3A is a schematic view of an apparatus according to another embodiment producing quantum entanglement between a substance and a medium such as water, and the medium subsequently producing non-local effect of the said substance on a biological system after it is consumed by the biological system.

FIG. 3B is a schematic view of another configuration of the apparatus illustrated in FIG. 3A producing quantum entanglement between a substance and a medium such as water, and the medium subsequently producing non-local effect of said substance on a biological system after it is consumed by the biological system.

FIG. 4A is a schematic view of three embodiments of an apparatus in the present invention for producing quantum entanglement between a first and second medium such as two parts of water.

FIG. 4B is a schematic view of another three embodiments of an apparatus in the present invention for producing quantum entanglement within a medium such as water.

FIG. 5A is a schematic view of a method according to one embodiment for using two parts of a quantum entangled medium such as water for transmitting a beneficial effect of a substance or an encoded message from one location to another.

FIG. 5B is a schematic view of a method according to another embodiment for using two parts of a quantum entangled medium for transmitting a beneficial effect of a substance or an encoded message from one location to another.

FIG. 5C is a schematic view of a method according to yet another embodiment for using two parts of a quantum entangled medium for quantum entangling two biological systems such as two human bodies for beneficial purposes.

FIG. 6 is a schematic view of a method according to one embodiment for producing quantum entanglement between two biological systems such as two human bodies for beneficial purposes.

FIG. 7 is a schematic view of a method according to another embodiment for using two parts of a quantum entangled medium to transmit a beneficial effect of a substance or an encoded message from one location to another.

FIG. 8A is a schematic view of a method according to one embodiment for objectively and quantitatively detecting and measuring non-local effects in biological systems such as a human.

FIG. 8B is a schematic view of another method according to another embodiment for objectively and quantitatively detecting and measuring non-local effects in biological systems such as a human.

FIG. 8C is a schematic view of yet another method according to yet another embodiment for objectively and quantitatively detecting and measuring non-local effects in biological systems such as a human.

FIGS. 9A, 9B, 9C, 9D, 9E, 9F, 9G and 9H are chart views of five sets of experimental data showing time series of heart rates obtained on a test subject under blind conditions according to embodiment illustrated in FIG. 7 and FIG. 8 for objectively and quantitatively detecting and measuring a non-local effect.

FIGS. 10A and 10B are chart views of two sets of experimental data showing time series of heart rates obtained on another subject under blind conditions according to embodiment illustrated in FIG. 7 and FIG. 8 for objectively and quantitatively detecting and measuring a non-local effect.

### DETAILED DESCRIPTION OF THE INVENTION

The apparatus of the present invention in one broad embodiment includes a quantum entanglement generating source, a substance disposed adjacent to the said source and a container holding the said substance.

The apparatus of the present invention in another broad embodiment also includes a detecting device for objectively and quantitatively detecting and measuring non-local effects in biological systems such as a human.

The said source will be, depending on a particular use, any source, such as a magnetic coil connected to a driving device, laser, microwave oven, flashlight or even a biological system, which is capable of generating quantum-entangling members such as photons, electrons, atoms or molecules when said source operates. The selection and operating specifications of the source will vary according to the use. The person skilled in the art will be able readily to determine the appropriate source and operating specifications of said source, with only routine experimentation, for optimum performance of the specific use intended.

The said substance will be, depending on the use, a single substance or a mixture of several substances and has the physical forms of a liquid, gel, powder, solid or gas, or a mixture of these said forms. Again, the selection of the substance or specific mixture of substances and their precise concentrations will vary according to the use. It will, however, from the information herein, be well within the ability of a person of ordinary skill in the art to select the appropriate mixture of substances for the particular use intended by such person, with no more than routine experimentation.

The container will be any material and form capable of supportive functions such as a simple plastic frame, a glass or plastic bottle, or polymer matrix. The container will be optional if the substance or the mixture of substances will be made into an appropriate solid. Further, the container will be at least partially transparent to quantum-entangling members such as photons generated by the source.

The said detecting device will be, depending on a particular use, any detecting device, such as heart rate monitor, blood pressure monitor, electroencephalogram (EEG) machine, magnetoencephalogram (MEG) machine or even a biophoton detector, which is capable of detecting and measuring physical, chemical and/or biological non-local effects such as changes in heart rate, blood pressure, EEG, MEG and or biophoton emissions. The selection and operating specifications of the detecting device will vary according to the use. The person skilled in the art will be able readily to determine the appropriate detecting device and operating specifications of said detecting device, with only routine experimentation, for optimum performance of the specific use intended.

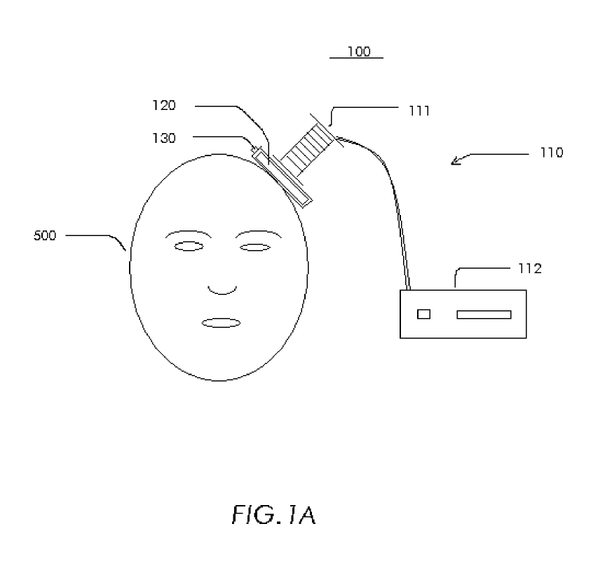

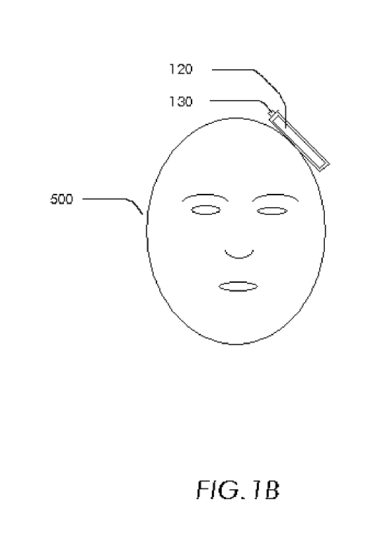

Considering first FIG. 1A, the apparatus 100 of the present invention in one embodiment includes a magnetic coil 111 connected to an audio system 112 as the generating source 110, a substance 120 disposed adjacent to the said source 110 and a container 130 holding the said substance 120.

In one particular embodiment, the container 130 is a small glassware of the dimensions about 1″×3″×4″ with a useful internal volume of about 20 ml, and the source 110 is made up of a magnetic coil 111 and an audio system 112 connected to the said magnetic coil. The said small glassware has a cap which is removable so that the container can be filled or emptied. The said magnetic coil is made up of a 75-feet and 26-gauge magnetic wire coated with enamel for insulation and wound on an open-ended plastic tube of the dimensions 3″ in length and 1.5″ in diameter. The said audio system is a typical consumer electronic product or a combination of several consumer electronic products readily available from a consumer electronics store.

When music is played on the audio system and the magnetic coil is connected to the speaker output of the audio system, the magnetic coil produces magnetic pulses with frequencies in the range of 0 Hz to 10 kHz and output in the range of 0 Watt to more than 50 Watts depending on the audio output setting and the type of audio system is used. These values for frequency and output power of the magnetic pulses emitted from the magnetic coils can be adjusted for optimal performance of the particular use intended.

To use the apparatus having this particular embodiment, one disposes the said apparatus 100 adjacent to a responsive target 500 such as a person's brain, and plays music on the audio system 112 with a desired output power and for a desired length of time whereby the photons generated by the magnetic coil 111 first quantum-entangle with quantum entities inside the substance 120, then travel to the biological system 500 and subsequently entangle with the quantum entities inside the biological system 500 producing non-local effect of the substance 120 on the biological system 500 through quantum entanglement.

FIG. 1B illustrates one method of directly producing non-local effect of a substance on a biological system through photons emitted by the biological system itself. The essential steps include providing a substance 120 responsive to photons emitted by a biological system 500; disposing said substance 120 adjacent to said biological system 500 whereby said photons quantum-entangle with said substance which produces said non-local effect of said substance on said biological system.

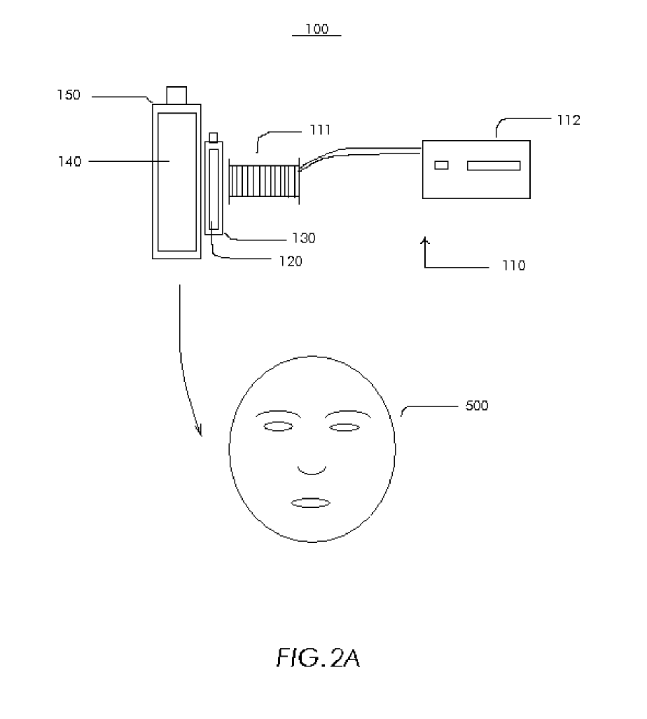

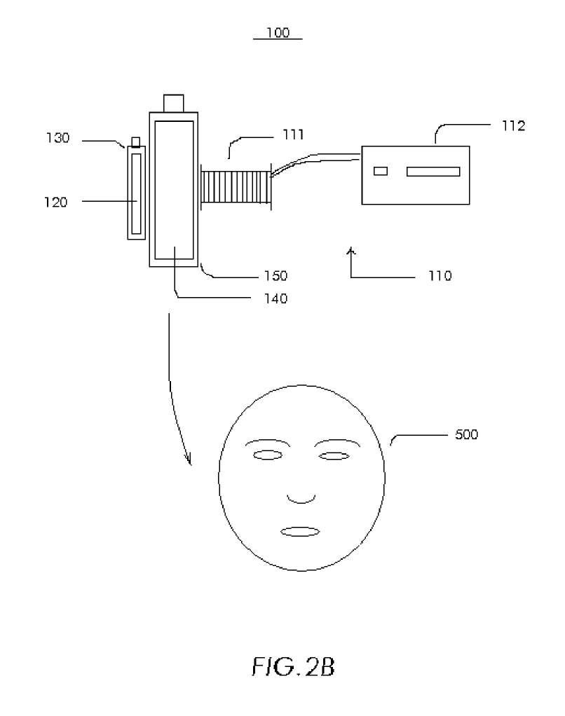

Considering next FIG. 2A, the apparatus 100 of the present invention includes a quantum entanglement generating source 110, a substance 120 disposed adjacent to the said source 110, a first container 130 holding the said substance 120, a medium 140 disposed adjacent to the substance 120, and a second container 150 holding the medium 140.

Again, the said first or second container will be, depending on a particular use, any material and form capable of supportive functions such as a simple frame, glassware, plastic ware or polymer matrix. The first or second container will be optional if the substance, the mixture of substances, medium or mixture of media will be made into an appropriate solid. Further, the first and second container will be at least partially transparent to quantum-entangling members such as photons generated by the source.

In one embodiment, the medium to be quantum-entangled the substance is an aqueous liquid, preferably water. It will be understood, however, that the invention is not limited only to quantum-entanglement of water with the substance but also applies to the quantum-entanglement of other medium with the substance.

In one particular embodiment, the second container is a large glassware of the dimensions 2″×8″×10″ with a useful internal volume of 200 ml, the medium is 200 ml tap water, and other elements of the apparatus are the same as the ones described in the particular embodiment of the apparatus shown in FIG. 1A. The said large glassware has a cap which is removable so that the second container can be filled or emptied.

To use the apparatus having this particular embodiment, one plays music on the audio system 112 with a desired output power and for a desired length of time whereby the photons generated by the magnetic coil 111 first entangle with quantum entities inside the substance 120, then travel to the medium 140 and subsequently entangle with quantum entities inside the medium 140 producing quantum entanglement between the substance 120 and the medium 140. Subsequently, to use the quantum entangled medium 140, one applies said medium, in one embodiment, to a biological system 500 such as a human body to produce non-local effect of the substance 120 on the said biological system 500.

Considering next FIG. 2B, the apparatus 100 of the present invention is a different configuration of the apparatus shown FIG. 2A in which the medium 140 is disposed between the substance 120 and the said source 110.

In one particular embodiment, all elements of the apparatus are the same as the ones in the particular embodiment of the apparatus shown in FIG. 2A. To use the apparatus having this particular embodiment, one again plays music on the audio system 112 with a desired output power and for a desired length of time such that the photons produced by the magnetic coil 111 first entangle with quantum entities inside the medium 140, then travel to the substance 120 and subsequently entangle with quantum entities inside the substance 120 producing quantum entanglement between the medium 140 and the substance 120. Subsequently, to use the quantum entangled medium 140, one applies said medium, in one embodiment, to a biological system 500 such as the human body to produce non-local effect of the substance 120 on the said biological system 500.

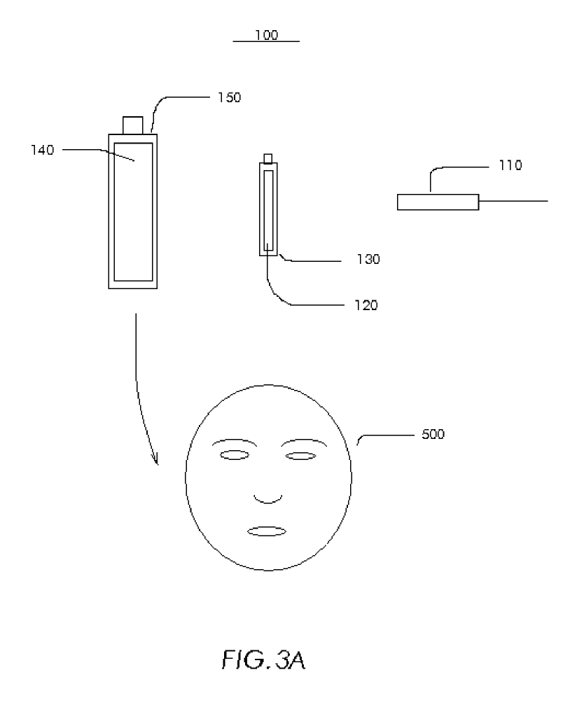

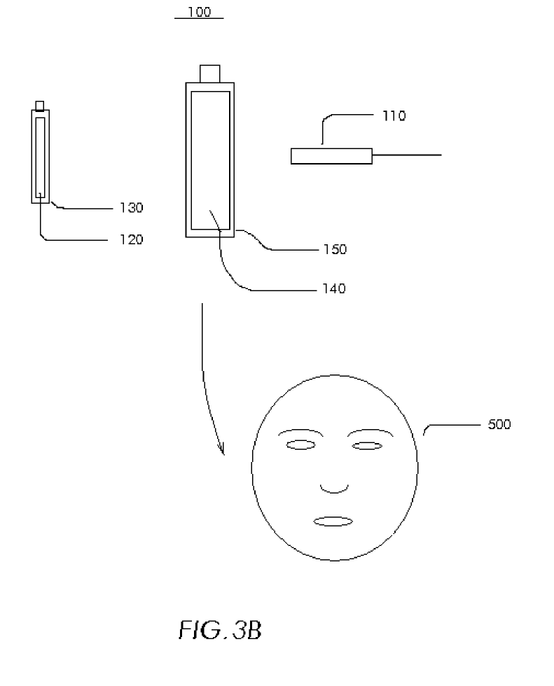

Considering next FIG. 3A, the apparatus 100 of the present invention includes a quantum entanglement generating source 110, a substance 120 disposed adjacent to the said source at a first adjustable distance, a first container 130 holding the said substance 120, a medium 140 disposed adjacent to the substance 120 at a second adjustable distance, and a second container 150 holding the medium 140.

In one particular embodiment, the quantum entanglement generating source 110 is a laser with a 50 mW output and wavelengths in the ranges of 635 nm-675 nm, and other elements of the apparatus are the same as the ones described in the particular embodiment of the apparatus illustrated in FIG. 2A. These values for frequency and wavelength of the laser light emitted from the laser can be adjusted for optimal performance of the particular use intended.

To use the apparatus having this particular embodiment, one operates the laser 110 with a desired output power and for a desired length of time whereby the photons generated by the laser 110 first entangle with quantum entities inside the substance 120, then travel to the medium 140 and subsequently entangle with quantum entities inside the medium 140 producing quantum entanglement between the substance 120 and the medium 140. Subsequently, to use the quantum entangled medium 140, one applies said medium, in one embodiment, to a biological system 500 such as the human body to produce non-local effect of the substance 120 on the said biological system 500.

Considering next FIG. 3B, the apparatus 100 of the present invention is a different configuration of the apparatus shown FIG. 3A in which the medium 140 is disposed between the substance 120 and the said source 110.

In one particular embodiment, all elements of the apparatus are the same as the ones in the particular embodiment of the apparatus shown in FIG. 3A. To use the apparatus having this particular embodiment, one again operates the laser 110 with a desired output power and for a desired length of time whereby the photons generated by the laser 110 first entangle with quantum entities inside the medium 140, then travel to the substance 120 and subsequently entangle with quantum entities inside the substance 120 producing quantum entanglement between the medium 140 and the substance 120. Subsequently, to use the quantum entangled medium 140, one applies said medium, in one embodiment, to a biological system 500 such as the human body to produce non-local effect of the substance 120 on the said biological system 500.

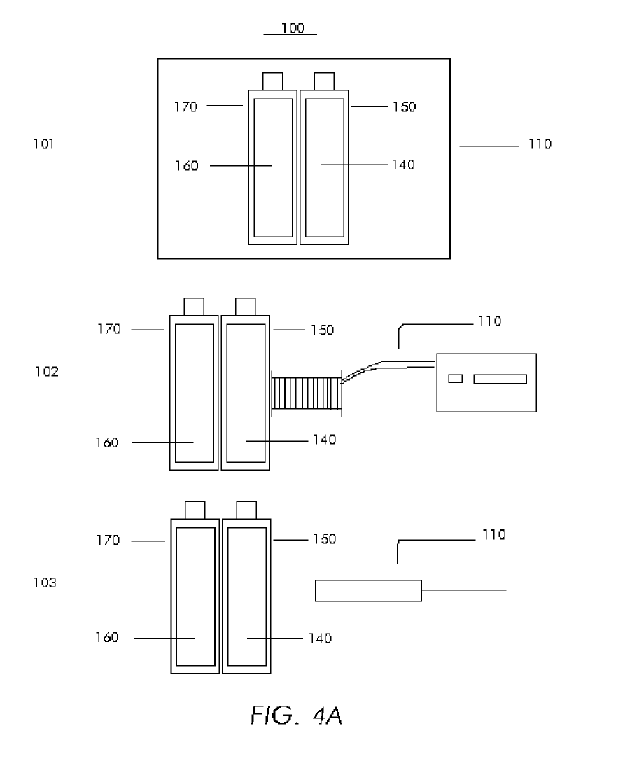

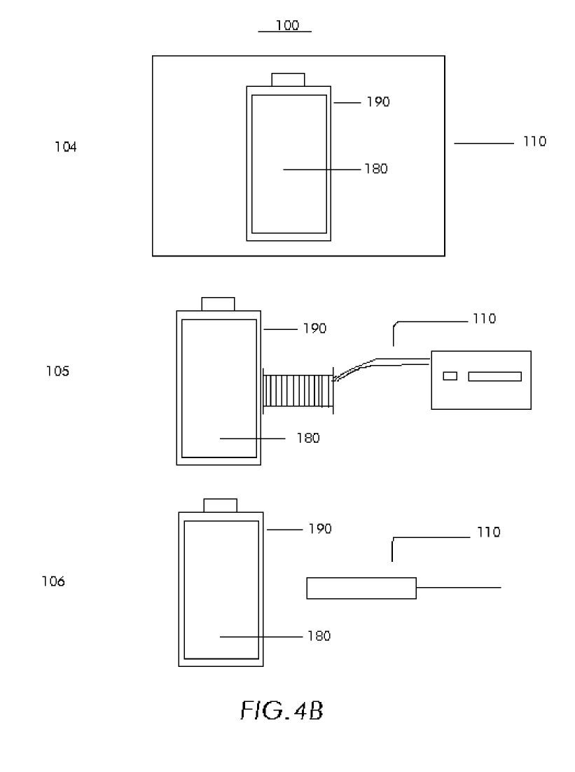

Considering next FIG. 4A, the apparatus 100 of the present invention includes a quantum entanglement generating source 110, a first medium 140, a first container 150 holding the said first medium 140, a second medium 160 disposed adjacent to the first medium 140, and a second medium container 170 holding the second medium 160.

The said first or second container will be, depending on a particular use, any material and form capable of supportive functions such as a simple frame, glassware, plastic ware or polymer matrix. The first or second container will be optional if the first or second medium will be made into an appropriate solid. Further, the first and second container will be at least partially transparent to quantum-entangling members such as photons generated by the source.

In one embodiment, the first and second medium to be quantum-entangled with each other are both an aqueous liquid, preferably water. It will be understood, however, that the invention is not limited only to quantum-entanglement of water with water but also applies to the quantum-entanglement of other media with each other.

In one embodiment 101, the said source 110 is a microwave oven enclosing the said medium 140 and 160 disposed adjacent to each other. In another embodiment 102, the said source 110 is made up of a magnetic coil 111 and an audio system 112 connected to the said magnetic coil with the said magnetic coil disposed adjacent to medium 140. In yet another embodiment 103, the said source 110 is a laser disposed adjacent to medium 140.

To use each apparatus 101, 102 or 103 having the respective embodiment, one operates the particular quantum entanglement source 110 with a desired output power and for a desired length of time whereby the photons generated by the said source 110 first entangle with quantum entities inside the medium 140, then travel to the medium 160 and subsequently entangle with quantum entities inside the medium 160 producing quantum entanglement between medium 140 and 160.

Considering next FIG. 4B, the apparatus 100 of the present invention includes a quantum entanglement generating source 110, a medium 180, and a container 190 holding the said medium 180. The said apparatus is essentially a different configuration of the apparatus illustrated in FIG. 4A.

Again, in one particular embodiment 104, the said source 110 is a microwave oven enclosing the medium 180. In another particular embodiment 105, the said source 110 is made up of a magnetic coil 111 and an audio system 112 connected to the said magnetic coil with the said magnetic coil disposed adjacent to the medium 180. In yet another embodiment 106, the said source 110 is a laser disposed adjacent to the medium 180.

To use each apparatus 104, 105 or 106 having the respective embodiment, one operates the quantum entanglement source 110 with a desired output power and for a desired length of time whereby the photons generated by the said source 110 first entangle with some quantum entities inside the medium 180, and second entangle with some other quantum entities inside the same medium 180 producing quantum entanglement within the medium 180. Subsequently, to use the quantum-entangled medium 180, the said medium is divided into two or more parts.

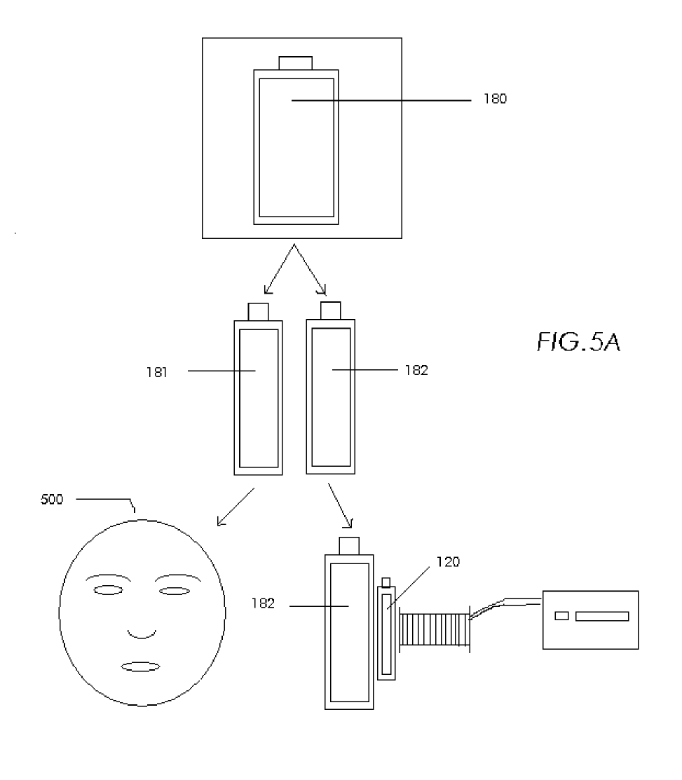

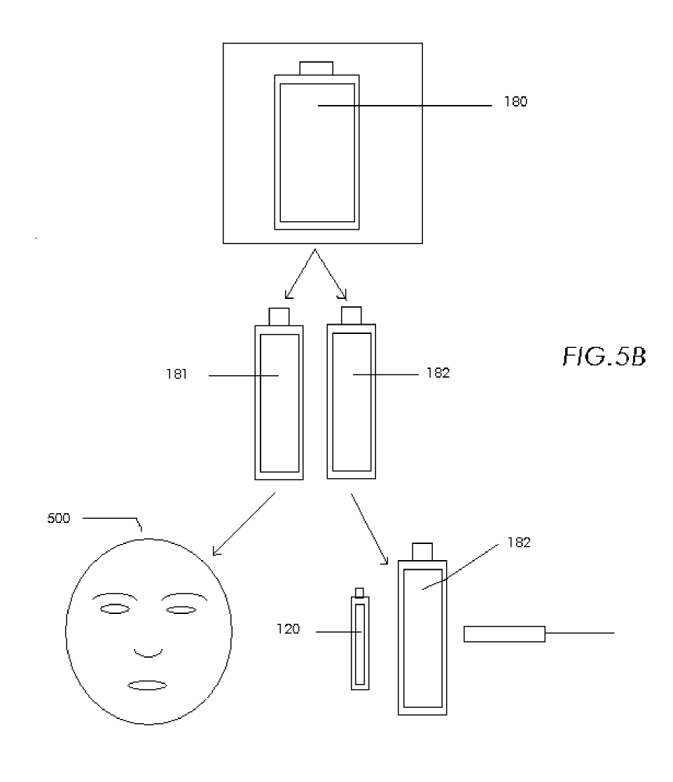

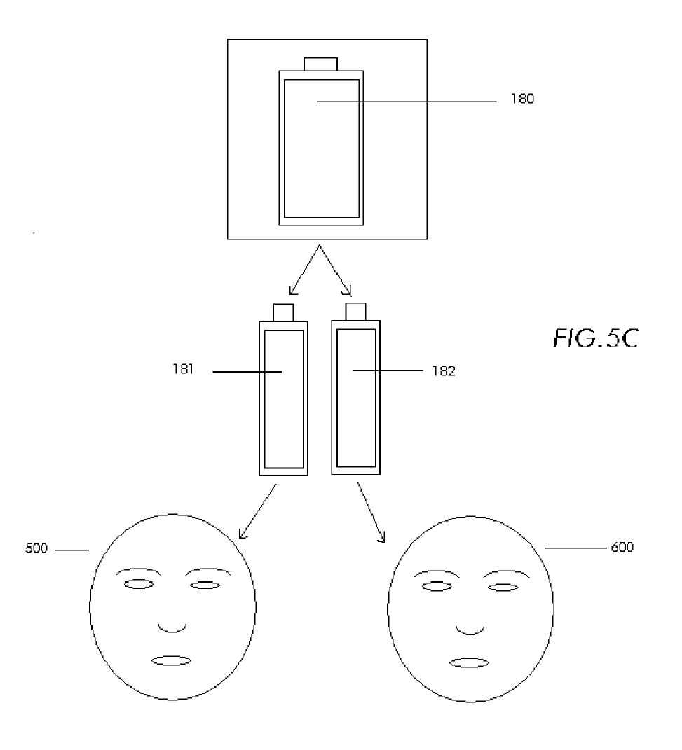

FIG. 5A illustrates one method of beneficially using two parts 181 and 182 of a quantum-entangled medium 180 produced with apparatus 104, 105 or 106 illustrated in FIG. 4B (or 140 and 160 produced with apparatus 101, 102 or 103 illustrated in FIG. 4A). The essential steps include providing two parts 181 and 182 of a quantum-entangled medium 180, applying one part 181 to a biological system 500 such as the human body, and quantum entangling the other part 182 with a desired substance 120 such as a particular medication or substance encoded with a message whereby non-local effect of the substance 120 on the said biological system 500 is produced for a beneficial purpose.

FIG. 5B illustrates another method of beneficially using two parts 181 and 182 of a quantum-entangled medium 180 produced with apparatus 101, 102 or 103 illustrated in FIG. 4B (or 140 and 160 produced with apparatus 101, 102 or 103 illustrated in FIG. 4A). The essential steps are the same as those described immediately above.

FIG. 5C illustrates yet another method of beneficially using two parts 181 and 182 of a quantum-entangled medium 180 produced with apparatus 101, 102 or 103 illustrated in FIG. 4B (or 140 and 160 produced with apparatus 101, 102 or 103 illustrated in FIG. 4A). The essential steps include providing two parts 181 and 182 of a quantum-entangled medium 180, applying one part 181 to a biological system 500 such as a human body and other part 182 to another biological system 600 such as another human body whereby the two biological systems 500 and 600 are quantum entangled for a beneficial purpose.

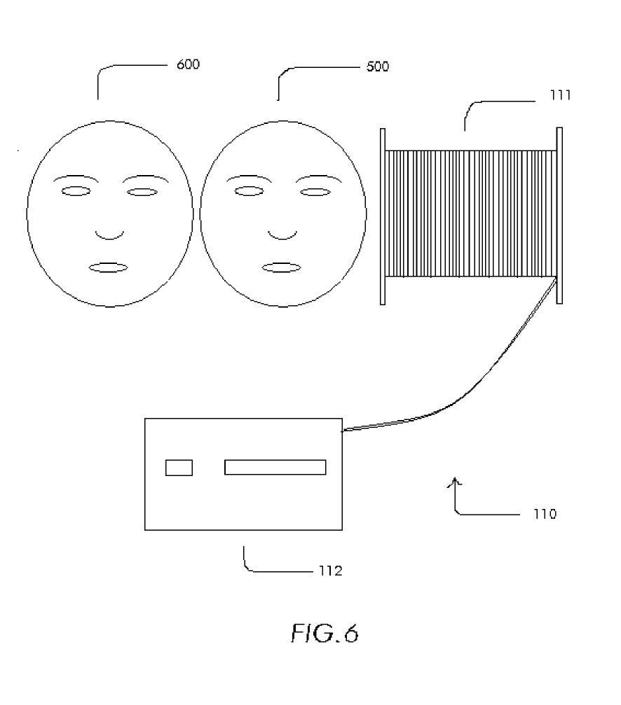

FIG. 6 illustrates a method of directly quantum entangling two biological systems 500 and 600 such as two human brains for a beneficial purpose. The essential steps include providing a quantum entanglement source 110 such as a large magnetic coil 111 connected to a audio system 112 with high output power, disposing the biological systems 500 adjacent to the said source and the biological system 600 adjacent to the biological systems 500, playing music on the audio system 112 with a desired output power and for a desired length of time such that the photons generated by the magnetic coil 111 first entangle with quantum entities inside the biological system 500, then travel to biological system 600 and subsequently entangle with the quantum entities inside the biological system 600 producing quantum entanglement between the biological system 500 and 600.

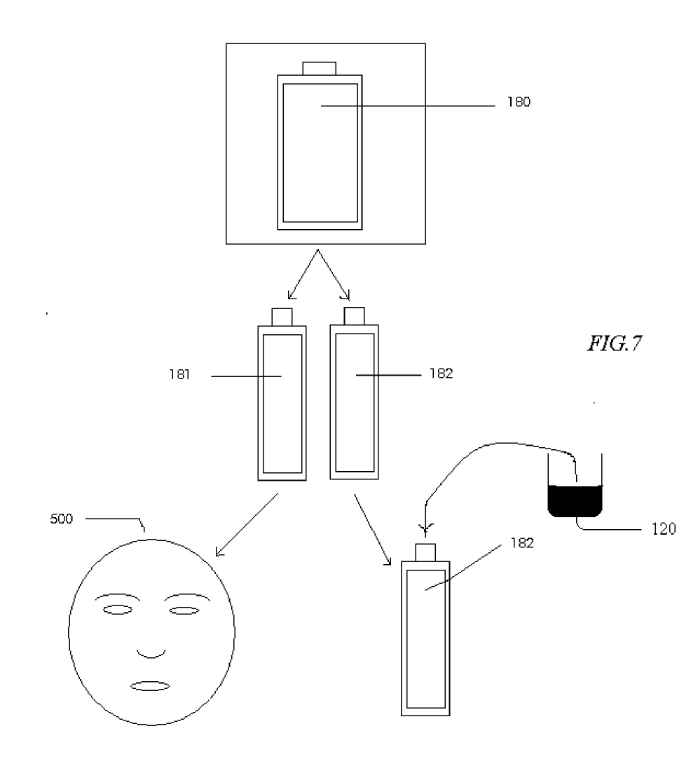

FIG. 7 illustrates one method of beneficially using two parts 181 and 182 of a quantum-entangled medium 180 produced with apparatus 104, 105 or 106 illustrated in FIG. 4B (or 140 and 160 produced with apparatus 101, 102 or 103 illustrated in FIG. 4A). The essential steps include providing two parts 181 and 182 of a quantum-entangled medium 180, applying one part 181 to a biological system 500 such as a human, and contacting the other part 182 with a desired substance 120 such as a particular medication or substance encoded with a message whereby non-local effect of the substance 120 on the said biological system 500 is produced for a beneficial purpose.

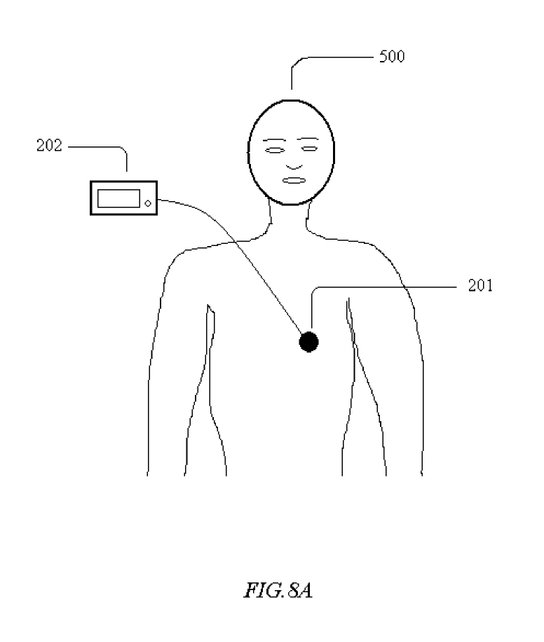

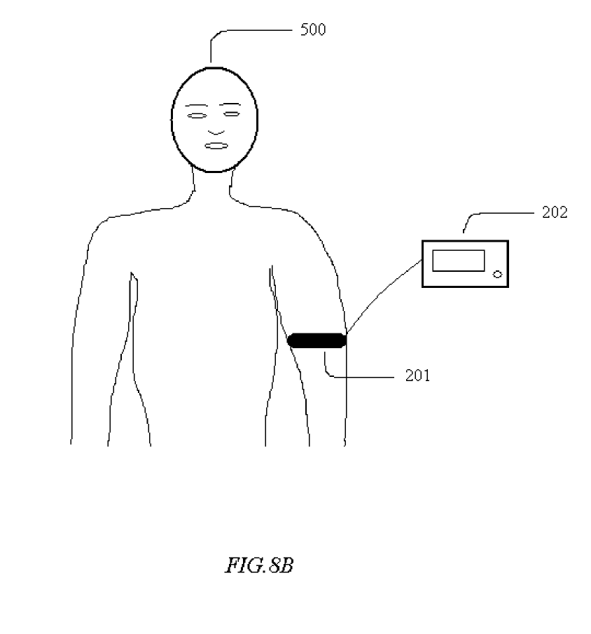

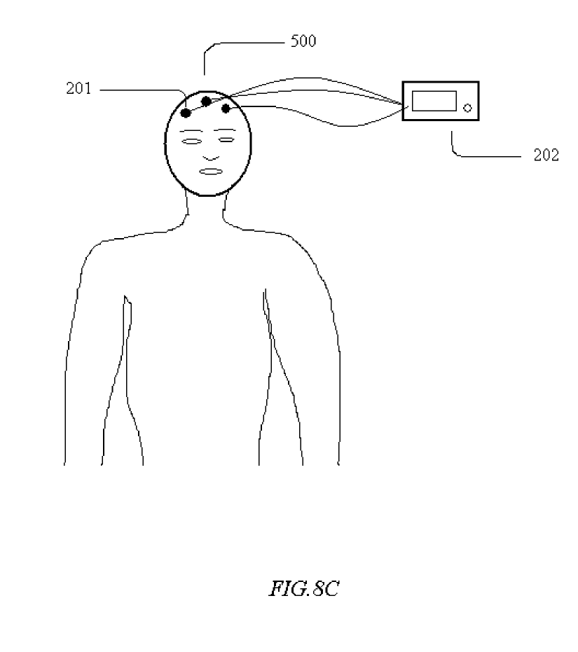

FIG. 8A illustrates one method of using a detecting device for objectively and quantitatively detecting and measuring a non-local effect in a biological system 500 such as a human. The essential steps include providing a detecting device (such as a heart rate monitor) comprising a probe 201 attached to the biological system 500 (such as chest area of the human) and a display mechanism 202 connected to said probe 201, or a wireless probe plus transmitter 201 attached to the biological system 500 (such as chest area of the human) and a wireless receiver plus display mechanism 202, and detecting a change of a physical, chemical or biological parameter (such as heart rate of the human) produced through quantum entanglement.

FIG. 8B illustrates a method of using another detecting device for objectively and quantitatively detecting and measuring a non-local effect in a biological system 500 such as a human. The essential steps include providing a detecting device (such as a blood pressure monitor) comprising a probe 201 attached to the biological system 500 (such as upper arm area of the human) and a display mechanism 202 connected with said probe 201, and detecting a change of a physical, chemical or biological parameter (such as blood pressure of the human) produced through quantum entanglement.

FIG. 8C illustrates a method of using yet another detecting device for objectively and quantitatively detecting and measuring a non-local effect in a biological system 500 such as a human. The essential steps include providing a detecting device (such as an EEG or MEG machine) comprising a plurality of probes 201 attached to the biological system 500 (such as head area of the human) and a display mechanism 202 connected with said probes 201, and detecting a change of a physical, chemical or biological parameter (such as EEG or MEG of the human) produced through quantum entanglement.

It will be appreciated that the particular features of the methods and apparatuses illustrated and described herein may be employed separately or in combination in any suitable manner so as to enhance the beneficial purposes. Those skilled in the art will also of course recognize that substitutions can be made, as long as the changes do not materially affect the ability of the methods and apparatuses disclosed herein.

Various experimental studies with the apparatus and methods disclosed herein were carried out to evaluate the quantum entanglement produced and the effects of various substances on responsive targets such as biological and/or chemical systems, and to verify that the said effects were non-local effects of the said substances produced through quantum entanglement.

Additional experimental studies with the detecting device disclosed herein were carried out to detect and measure objectively and quantitatively non-local effects of various substances in biological systems such a human.

In the first set of experiments, the apparatus illustrated in FIG. 1A having the particular embodiment described previously was used. Said apparatus included the magnetic coil with an estimated 20 W output disposed at one inch above the right side of a test subject's forehead, the small glassware inserted between the said coil and the forehead, a substance filling the said container, and the audio system with adjustable output power and frequency spectrum controls. When music was played on the audio system, the said magnetic coil produces magnetic pulses with frequencies in the range of 0 Hz to 10 kHz. Experiments were conducted with said container being filled with different general anesthetics, medications, or nothing/water as control, and the test subject being exposed to the magnetic pulses for 10 min and not being told the content in the said container or details of the experiments.

The indicators used to measure the biological and/or chemical effects of said treatment were the first-person experiences of any unusual sensations such as numbness, drowsiness and/or euphoria which the subject felt after the treatment and the relative degrees of these unusual sensations on a scale of 10 with 0=nothing, 1=weak, 2=light moderate, 3=moderate, 4=light strong, 5=strong, 6=heavily strong, 7=very strong, 8=intensely strong, 9=extremely strong and 10=intolerable. The duration of the unusual sensations and other symptoms after the treatment such as nausea, fatigue or headache were also recorded.

In the second set of experiments, the apparatus illustrated in FIG. 2A have the particular embodiment described previously was first used. The said apparatus included the magnetic coil, the large glassware with 200 m/fresh tap water, the small glassware inserted between the magnetic coil and large glassware, and a substance filling the said small glassware. Next, the apparatus illustrated in FIG. 3A have the particular embodiment described previously was used. The said apparatus included, among other elements described previously, the laser with a 50 mW output and wavelengths in the ranges of 635 nm-675 nm.

All Experiments in the second set were conducted in the dark with the small glassware being filled with different general anesthetics, medications, or nothing/water as control, the large glassware being filled with 200 m/tap water and exposed to the magnetic pulses or laser light for 30 min and the test subject consuming the treated tap water but not being told anything about the experiments. The indicators used for measuring the biological and/or chemical effects were the same as those used in the first set of experiments.

In addition, the second set of experiments was also carried out respectively with a 1200 W microwave oven and a flashlight powered by two size-D batteries. When the microwave oven was used, a glass tube containing 20 ml fresh tap water was submerged into a larger glass tube containing 50 ml general anesthetic and exposed to microwave radiation for 5 sec. The said procedure was repeated four times to collect a total of 200 ml treated tap water for consumption. When the flashlight was used, the magnetic coil shown in FIG. 2A was replaced with the flashlight.

To verify that the biological and/or chemical effects experienced by the test subjects were due to quantum entanglement between the quantum entities inside the test subjects and those in the substances under study, the following several sets of additional experiments were carried out.

In the first set of entanglement verification experiments, the apparatus as illustrated in FIG. 3B, having the particular embodiment described previously was used. When said apparatus operated, the laser light from the laser first passed through the large glassware filled with 200 ml tap water and then through the small glassware filled with a substance or nothing/water as control located about 300 cm away. To prevent reflected laser light from re-entering the large glassware holding the medium, the small glassware filled with a substance or nothing/water as control was positioned with an angle to the incoming laser light. After 30 min exposure to the laser light, a test subject consumed the treated tap water without being told the details of the experiments and report the biological and/or chemical effects felt for the next several hours.

In the second set of entanglement verification experiments, 400 ml tap water in the glassware illustrated in FIG. 4B was first exposed to the radiation of the magnetic coil with a 20 Watt output for 30 min or that of microwave oven with 1500 Watt output for 2 min. Then a test subject immediately consumed one-half of the water so exposed as shown in FIG. 5A or FIG. 5B. After 30 min from the time of consumption the other half was exposed to magnetic pulses as shown in FIG. 5A or the laser light for 30 minutes as shown in FIG. 5B. The test subject reported, without being told any details of the experiments, the biological and/or chemical effects felt for the whole period from the time of consumption to several hours after the exposure had stopped.

In the third set of entanglement verification experiments, one-half of 400 ml bottled Poland Spring water with a shelve time of at least three months was immediately consumed by a test subject. After 30 min from the time of consumption the other half was exposed to the magnetic pulses or laser light for 30 min using the apparatus shown in FIG. 5A and FIG. 5B respectively, and the test subject reported, without being told any details of the experiments, the biological and/or chemical effects felt for the whole period from the time of consumption to several hours after the exposure had stopped.

In the fourth set of entanglement verification experiments, a test subject took one-half of the 400 ml microwave or magnetic coil exposed water as shown in FIG. 4B to his/her workplace located more than 50 miles away (in one case to Beijing located more than 6,500 miles away) and consumed the same at the workplace at a specified time. After 30 min from the time of consumption, the other half was exposed to the magnetic pulses or the laser light for 30 min at the original location using the apparatus shown in FIG. 5A or FIG. 5B. The test subject reported any biological and/or chemical effects felt without knowing the details of the experiments for the whole period from the time of consumption to several hours after the exposure had stopped.

In the set of experiments for objectively and quantitatively detecting and measuring non-local effects in biological systems such as a human, the method and apparatus illustrated in FIG. 7 and FIG. 8A have the particular embodiment described previously and further below were used. The experiments were conducted under blind conditions in that the voluntary test subject did not know any detail of the experiment and the detecting and measuring person did not know the exact time when a substance was added.

To prepare for the experiments, five (5) tablets of Primatene (containing a total of 60 mg ephedrine, a heart stimulant) were crushed into powder and dissolved into 10 ml water; and 400 ml Poland Spring water in a plastic ware with a shelf time of at least three months was exposed to the radiation of microwave oven with a 1500 Watt output for 1 min as illustrated in FIG. 7. Primatene is an over-the-counter medication for asthma.

The test subject then immediately consumed one-half of the water so exposed as described above. After 30 min from the time of consumption, the 10 ml solution of Primatene was added into the other half of the microwaved water by a person in a different room about 50 feet away from the test subject and at a time not known by the test subject or the person measuring and recording the heart rate.

The time series of heart rate were measured as shown in FIG. 8A with a Polar FT4 wireless heart rate monitor and recorded by hand at the interval of every 0.1 min (6 sec) before, during and after Primatene was added into the other half of the microwaved water.

Table 1 summarizes the results obtained from the first two sets of experiments described above:

TABLE 1

| | 1st Set: | | 2nd Set: | | | | | | | |
|---|---|---|---|---|---|---|---|---|---|---|
| | Magn. Coil | | Magn. Coil | | Laser Light | | Flashlight | | Microwave | |
| | Test # | Effect | Test # | Effect | Test # | Effect | Test # | Effect | Test # | Effect |
| Anaesthetics | | | | | | | | | | |
| Subject A | 13 | Yes | 16 | Yes | 22 | Yes | 8 | Yes | 3 | Yes |
| Subject B | 2 | Yes | 2 | Yes | 3 | Yes | 0 | N/A | 1 | Yes |
| Subject C | 2 | Yes | 6 | Yes | 6 | Yes | 0 | N/A | 1 | Yes |
| Subject D | 2 | Yes | 1 | Yes | 5 | Yes | 0 | N/A | 0 | N/A |
| Medications | | | | | | | | | | |
| Subject A | 17 | Yes | 14 | Yes | 16 | Yes | 1 | Yes | 3 | Yes |
| Subject B | 1 | Yes | 1 | Yes | 3 | Yes | 0 | N/A | 2 | Yes |
| Subject C | 3 | Yes | 1 | Yes | 4 | Yes | 0 | N/A | 1 | Yes |
| Subject D | 0 | N/A | 0 | N/A | 3 | Yes | 0 | N/A | 1 | Yes |
| Control | | | | | | | | | | |
| Subject A | 12 | No | 5 | No | 11 | No | | | | |
| Subject B | 3 | No | 0 | N/A | 1 | No | | | | |
| Subject C | 1 | No | 2 | No | 4 | No | | | | |
| Subject D | 0 | N/A | 0 | N/A | 1 | No | | | | |

Table 2 breakdowns the summary in Table 1 into each general anesthetic plus morphine in the case of medications:

TABLE 2

| | 1st Set: | | 2nd Set: | | | | | | | |
|---|---|---|---|---|---|---|---|---|---|---|
| | Magn. Coil | | Mag. Coil | | Red Laser | | Flashlight | | Microwave | |
| | Test # | Effect | Test # | Effect | Test # | Effect | Test # | Effect | Test # | Effect |
| Chloroform | | | | | | | | | | |
| Subject A | 2 | Yes | 2 | Yes | 5 | Yes | 2 | Yes | 3 | Yes |
| Subject B | 0 | N/A | 0 | N/A | 1 | Yes | 0 | N/A | 1 | Yes |
| Subject C | 1 | Yes | 2 | Yes | 3 | Yes | 0 | N/A | 1 | Yes |
| Subject D | 1 | Yes | 0 | N/A | 2 | Yes | 0 | N/A | 0 | N/A |
| Chloroform D | | | | | | | | | | |
| Subject A | 3 | Yes | 2 | Yes | 2 | Yes | 1 | Yes | | |
| Subject B | 1 | Yes | 0 | N/A | 1 | Yes | 0 | N/A | | |
| Subject C | 0 | N/A | 0 | N/A | 1 | Yes | 0 | N/A | | |
| Subject D | 0 | N/A | 0 | N/A e | 0 | N/A | 0 | N/A | | |
| Isoflurance | | | | | | | | | | |
| Subject A | 3 | Yes | 6 | Yes | 5 | Yes | 4 | Yes | | |
| Subject B | 0 | N/A | 1 | Yes | 0 | N/A | 0 | N/A | | |
| Subject C | 0 | N/A | 1 | Yes | 1 | Yes | 0 | N/A | | |
| Subject D | 1 | Yes | 1 | Yes | 1 | Yes | 0 | N/A | | |
| Diethyl Ether | | | | | | | | | | |
| Subject A | 5 | N/A | 6 | Yes | 10 | Yes | 1 | Yes | | |
| Subject B | 1 | N/A | 1 | Yes | 1 | Yes | 0 | N/A | | |
| Subject C | 1 | N/A | 3 | Yes | 1 | Yes | 0 | N/A | | |
| Subject D | 0 | N/A | 0 | N/A | 2 | Yes | 0 | N/A | | |
| Morphine | | | | | | | | | | |
| Subject A | 5 | Yes | 7 | Yes | 5 | Yes | | | | |
| Subject B | 0 | N/A | 1 | Yes | 2 | Yes | | | | |
| Subject C | 0 | N/A | 1 | Yes | 2 | Yes | | | | |
| Subject D | 0 | N/A | 0 | N/A | 2 | Yes | | | | |
| Other Med | | | | | | | | | | |
| Subject A | 7 | Yes | 4 | Yes | | | | | | |
| Subject B | 1 | Yes | 0 | N/A | | | | | | |
| Subject C | 3 | Yes | 0 | N/A | | | | | | |
| Subject D | 0 | N/A | 0 | N/A | | | | | | |

With respect to the test subjects, all four voluntarily consented to the proposed experiments. To ensure safety, all initial experiments were conducted on the inventor, Subject A, by himself. Further, all general anaesthetics used in the study were properly obtained for research purposes and all medications were either leftover items originally prescribed to Subject C's late mother or items available over the counter. To achieve proper control, repeating experiments on Subject A were carried out by either Subject B or C in blind settings, that is, he was not told whether or what general anaesthetic or medication were applied before the end of the experiments. Further, all experiments on Subject B, C and D were also carried out in blind settings, that is, these test subjects were not told about the details of the experiments on them or whether or what general anaesthetic or medication were applied.

As shown in Table 1, in the control studies for the first set of experiments all test subjects did not feel anything unusual from the exposure to magnetic pulses except vague or weak local sensation near the site of exposure. In contrast, all general anaesthetics studied produced clear and completely reproducible biological and/or chemical effects such as various brain effects in various degrees and durations as shown in Table 2 as if the test subjects had actually inhaled the same. These brain effects were first localized near the site of treatment and then would spread over the whole brain and fade away within several hours. But residual brain effects (hangover) would linger on for more than 12 hours in most cases. Among the general anaesthetics studied, chloroform and deuterated chloroform (chloroform D) produced the most pronounced and potent brain effects in both strength and duration followed by isoflorance and diethyl ether. Tribromoethanol dissolved in water (1:50 by weight) and ethanol also produced noticeable effects but they are not summarized in the table. Other biological and/or chemical effects included nausea, fatigue and numbness in various degrees.

As also shown in Table 1, while the test subjects did not feel anything unusual from consuming the tab water treated in the control experiments with magnetic pulses or laser light, all general anaesthetics studied produced clear and completely reproducible biological and/or chemical effects such as brain effects in various degrees and durations respectively as shown in Table 2 similar to the observations in the first set of experiments. These brain effects were over the whole brain, would first intensify within the first half hour after the test subjects consumed the treated water and then would fade away within the next a few hours. But residual brain effects would linger on for more than 12 hours as in the first set of experiments. Among the general anaesthetics studied, again chloroform and deuterated chloroform produced the most pronounced and potent effect in strength and duration followed by isoflorance and diethyl ether as illustrated in FIG. 5. In addition, available results with flashlight and microwave as photon sources are also summarized in Table 1 respectively. In both cases general anaesthetics tested produced clearly and reproducible brain effects with but the brain effects produced by microwave were much stronger than those by flashlight. Tribromoethanol dissolved in water (1:50 by weight) and ethanol also produced noticeable effects but they are not summarized in the table. Other biological and/or chemical effects included nausea, fatigue and numbness in various degrees.

Table 1 also summarizes results obtained with several medications including morphine, fentanyl, oxycodone, nicotine and caffeine in both first and second sets of experiments. It was found that they all produced clear and completely reproducible biological and/or chemical effects such as brain effects including euphoria and/or hastened alertness in various degrees and durations respectively. For example, in the case of morphine in the first set of experiments the brain effect was first localized near the site of treatment and then would spread over the whole brain and fade away within several hours. In the case of morphine in the second set of experiments the brain effect was over the whole brain, would first intensify within the first half hour after the test subjects consumed the treated water and then would fade away within the next a few hours.

Comparative experiments were also conducted on Subject A and C with chloroform and diethyl ether by asking them to inhale the vapours of chloroform and diethyl respectively for 5 sec and compare the biological and/or chemical effects such as brain effect felt with those in the two sets of experiments described above. The brain effects induced in these comparative experiments are qualitatively same as those produced in various experiments described above when chloroform and diethyl ether were used respectively.

Table 3 summarizes the results obtained with the entanglement verification experiments carried out so far with chloroform, deuterated chloroform, diethyl ether and morphine:

TABLE 3

| | First Set | | Second Set | | Third Set | | Fourth Set | |
|---|---|---|---|---|---|---|---|---|
| | Test # | Effect | Test # | Effect | Test # | Effect | Test # | Effect |
| Subject A | 8 | Yes | 8 | Yes | 3 | Yes | 3 | Yes |
| Subject B | 2 | Yes | 3 | Yes | 2 | Yes | 1 | Yes |
| Subject C | 3 | Yes | 2 | Yes | 1 | Yes | 1 | Yes |
| Control | | | | | | | | |
| Subject A | 2 | No | 8 | No | 3 | No | 3 | No |
| Subject B | 0 | N/A | 3 | No | 2 | No | 1 | No |
| Subject C | 1 | No | 2 | No | 1 | No | 1 | No |

With all four sets of entanglement verification experiments, clear and consistently reproducible biological and/or chemical effects such as brain effects were experienced by the test subjects above and beyond what were noticeable in the control portions of the experiments under blind settings. With respect to the second, third and fourth sets of entanglement verification experiments, the only possible explanation for the brain effects and other biological and/or chemical effects experienced by the test subjects are that these effects were the consequences of quantum entanglement because the water consumed by the test subjects was never directly exposed to the magnetic pulses or the laser lights in the presence of the substances under studies.

More specifically, in the first set of entanglement verification experiments, the biological and/or chemical effects such as brain effects experienced by the test subjects were the same as those in which the apparatus shown in FIG. 3A was used. In the second, third and fourth sets of these experiments, all test subjects did not feel anything unusual in the first half hour after consuming the first half of the water either radiated with microwave, magnetic pulses or just sit on the shelf for more than 3 months. But within minutes after the second half of the same water was exposed to the laser light or magnetic pulses in the presence of general anesthetics or morphine, the test subjects experienced clear and completely reproducible biological and/or chemical effects such as brain effects of various intensities as if they have actually inhaled the general anaesthetic used in the exposure of the second half of the water. The said brain effects would first intensify within minutes after the exposure began and persist for the duration of the said exposure and for the next several hours after the exposure had stopped. Other biological and/or chemical effects included nausea, fatigue and numbness in various degrees. Further, all other conditions being the same, the magnetic coil used produced more intense biological and/or chemical effects such as brain effects than the laser used. Furthermore, all other conditions being the same, when the water was first radiated with microwave or magnetic pulses before consumption it produced much more intense biological and/or chemical effects such as brain effects than the water just sitting on the shelve for more than 3 months before consumption.

There are other indications that quantum entanglement was the cause of the biological and/or chemical effects such as brain effects experienced by the test subjects. First, the biological and/or chemical effects inducing mean could not be transmitted through an electrical wire as reported above. Second, the said inducing mean do not depend on the wavelengths of the photons applied, thus mere interactions among the photons, a chemical substance and water will induce the biological and/or chemical effects such as brain effects after a test subject consumes the water so interacted.

Table 4 and Table 5 list five (5) sets of experimental data obtained on Subject B as described previously with each set comprising a test and baseline (control) time series of heart rate.

Table 4 shows the time series of heart rate with 10 ml solution of five (5) tablets of Primatene (containing 60 mg ephedrine) being added at the marked time of four (4) minute:

TABLE 4

| Time | Test1 | Test2 | Test3 | Test4 | Test5 | Mean | |
|---|---|---|---|---|---|---|---|
| 0.1 | 66. | 62. | 59. | 64. | 59. | 62.0 | |
| 0.2 | 66. | 62. | 59. | 64. | 59. | 62.0 | |
| 0.3 | 66. | 62. | 59. | 63. | 59. | 61.8 | |
| 0.4 | 66. | 62. | 60. | 63. | 58. | 61.8 | |
| 0.5 | 67. | 61. | 60. | 62. | 59. | 61.8 | |
| 0.6 | 67. | 60. | 60. | 61. | 61. | 61.8 | |
| 0.7 | 68. | 62. | 61. | 61. | 61. | 62.6 | |
| 0.8 | 68. | 62. | 60. | 62. | 60. | 62.4 | |
| 0.9 | 66. | 61. | 60. | 62. | 60. | 61.8 | |
| 1.0 | 67. | 61. | 61. | 61. | 59. | 61.8 | |
| 1.1 | 66. | 61. | 60. | 61. | 59. | 61.4 | |
| 1.2 | 66. | 61. | 59. | 62. | 59. | 61.4 | |
| 1.3 | 67. | 60. | 58. | 64. | 59. | 61.6 | |
| 1.4 | 67. | 60. | 58. | 64. | 59. | 61.6 | |
| 1.5 | 67. | 60. | 59. | 64. | 59. | 61.8 | |
| 1.6 | 67. | 61. | 58. | 64. | 60. | 62.0 | |
| 1.7 | 67. | 61. | 58. | 63. | 61. | 62.0 | |
| 1.8 | 67. | 61. | 59. | 63. | 60. | 62.0 | |
| 1.9 | 68. | 61. | 59. | 62. | 60. | 62.0 | |
| 2.0 | 67. | 61. | 59. | 62. | 60. | 61.8 | |
| 2.1 | 66. | 61. | 62. | 62. | 59. | 62.0 | |
| 2.2 | 66. | 61. | 62. | 62. | 61. | 62.4 | |
| 2.3 | 67. | 61. | 62. | 62. | 60. | 62.4 | |
| 2.4 | 67. | 62. | 63. | 62. | 60. | 62.8 | |
| 2.5 | 66. | 63. | 63. | 62. | 58. | 62.4 | |
| 2.6 | 66. | 61. | 63. | 63. | 58. | 62.2 | |
| 2.7 | 64. | 61. | 63. | 63. | 58. | 61.8 | |
| 2.8 | 64. | 61. | 63. | 63. | 58. | 61.8 | |
| 2.9 | 65. | 61. | 63. | 63. | 58. | 62.0 | |
| 3.0 | 65. | 62. | 63. | 63. | 59. | 62.4 | |
| 3.1 | 65. | 62. | 64. | 63. | 59. | 62.6 | |
| 3.2 | 65. | 61. | 64. | 63. | 60. | 62.6 | |
| 3.3 | 65. | 61. | 63. | 62. | 61. | 62.4 | |
| 3.4 | 65. | 61. | 63. | 63. | 61. | 62.6 | |
| 3.5 | 65. | 62. | 63. | 63. | 61. | 62.8 | |
| 3.6 | 65. | 62. | 63. | 64. | 62. | 63.2 | |
| 3.7 | 64. | 62. | 63. | 64. | 62. | 63.0 | |
| 3.8 | 64. | 62. | 64. | 64. | 62. | 63.2 | |
| 3.9 | 65. | 62. | 64. | 64. | 62. | 63.4 | |
| 4.0 | 65. | 63. | 63. | 63. | 61. | 63.0 | (Added Primatene |
| | | | | | | | Here) |
| 4.1 | 67. | 62. | 63. | 63. | 61. | 63.2 | |
| 4.2 | 68. | 63. | 63. | 64. | 61. | 63.8 | |
| 4.3 | 69. | 64. | 63. | 63. | 61. | 64.0 | |
| 4.4 | 70. | 64. | 63. | 62. | 61. | 64.0 | |
| 4.5 | 71. | 64. | 63. | 62. | 61. | 64.2 | |
| 4.6 | 72. | 64. | 63. | 63. | 60. | 64.4 | |
| 4.7 | 71. | 63. | 63. | 63. | 59. | 63.8 | |
| 4.8 | 71. | 62. | 63. | 63. | 59. | 63.6 | |
| 4.9 | 72. | 62. | 64. | 64. | 60. | 64.4 | |
| 5.0 | 71. | 62. | 64. | 64. | 60. | 64.2 | |
| 5.1 | 70. | 61. | 66. | 65. | 61. | 64.6 | |
| 5.2 | 70. | 61. | 66. | 66. | 61. | 64.8 | |
| 5.3 | 71. | 62. | 66. | 65. | 61. | 65.0 | |
| 5.4 | 71. | 61. | 66. | 65. | 61. | 64.8 | |
| 5.5 | 70. | 61. | 65. | 65. | 61. | 64.4 | |
| 5.6 | 70. | 61. | 64. | 64. | 61. | 64.0 | |
| 5.7 | 74. | 60. | 63. | 64. | 60. | 64.2 | |
| 5.8 | 74. | 61. | 63. | 63. | 61. | 64.4 | |
| 5.9 | 75. | 61. | 63. | 64. | 61. | 64.8 | |
| 6.0 | 75. | 61. | 64. | 66. | 61. | 65.4 | |
| 6.1 | 73. | 61. | 64. | 65. | 61. | 64.8 | |
| 6.2 | 73. | 61. | 66. | 64. | 61. | 65.0 | |
| 6.3 | 75. | 62. | 66. | 63. | 61. | 65.4 | |
| 6.4 | 75. | 63. | 66. | 65. | 62. | 66.2 | |
| 6.5 | 77. | 62. | 66. | 64. | 62. | 66.2 | |
| 6.6 | 77. | 62. | 65. | 63. | 62. | 65.8 | |
| 6.7 | 76. | 62. | 64. | 63. | 63. | 65.6 | |
| 6.8 | 76. | 63. | 63. | 63. | 64. | 65.8 | |
| 6.9 | 72. | 64. | 63. | 64. | 63. | 65.2 | |
| 7.0 | 72. | 64. | 65. | 64. | 62. | 65.4 | |
| 7.1 | 72. | 64. | 67. | 64. | 63. | 66.0 | |
| 7.2 | 72. | 64. | 68. | 64. | 63. | 66.2 | |
| 7.3 | 72. | 64. | 69. | 64. | 64. | 66.6 | |
| 7.4 | 70. | 63. | 68. | 64. | 64. | 65.8 | |
| 7.5 | 70. | 63. | 67. | 64. | 64. | 65.6 | |
| 7.6 | 70. | 63. | 67. | 64. | 63. | 65.4 | |
| 7.0 | 68. | 64. | 68. | 65. | 63. | 65.6 | |
| 7.8 | 68. | 64. | 70. | 65. | 62. | 65.8 | |
| 7.9 | 68. | 65. | 70. | 65. | 61. | 65.8 | |
| 8.0 | 68. | 65. | 66. | 65. | 61. | 65.0 | |
| 8.1 | 69. | 65. | 65. | 64. | 61. | 64.8 | |
| 8.2 | 69. | 65. | 65. | 63. | 60. | 64.4 | |
| 8.3 | 70. | 64. | 65. | 64. | 60. | 64.6 | |
| 8.4 | 70. | 63. | 66. | 65. | 61. | 65.0 | |
| 8.5 | 68. | 63. | 67. | 64. | 60. | 64.4 | |
| 8.6 | 68. | 62. | 66. | 62. | 60. | 63.6 | |
| 8.7 | 68. | 62. | 66. | 62. | 60. | 63.6 | |
| 8.8 | 68. | 63. | 65. | 62. | 60. | 63.6 | |
| 8.9 | 68. | 62. | 65. | 63. | 60. | 63.6 | |
| 9.0 | 68. | 62. | 65. | 63. | 60. | 63.6 | |
| 9.1 | 68. | 62. | 65. | 63. | 61. | 63.8 | |
| 9.2 | 68. | 62. | 65. | 63. | 61. | 63.8 | |
| 9.3 | 68. | 62. | 64. | 63. | 62. | 63.8 | |
| 9.4 | 68. | 60. | 64. | 64. | 63. | 63.8 | |
| 9.5 | 69. | 60. | 64. | 64. | 64. | 64.2 | |
| 9.6 | 69. | 60. | 65. | 65. | 63. | 64.4 | |
| 9.7 | 70. | 61. | 65. | 64. | 63. | 64.6 | |
| 9.8 | 70. | 62. | 65. | 65. | 63. | 65.0 | |
| 9.9 | 69. | 61. | 65. | 64. | 63. | 64.4 | |
| 10.0 | 69. | 61. | 63. | 64. | 62. | 63.8 | |

Table 5 shows the control data (baseline) obtained before any Primatene was added to the second half of the microwaved water (starting at 15 minute after Subject B consumed the first half of the microwaved water):

TABLE 5

| Time | Ctrl1 | Ctrl2 | Ctrl3 | Ctrl4 | Ctrl5 | Mean |
|---|---|---|---|---|---|---|
| 0.1 | 65. | 61. | 59. | 63. | 61. | 61.8 |
| 0.2 | 65. | 62. | 58. | 64. | 60. | 61.8 |
| 0.3 | 65. | 62. | 58. | 63. | 61. | 61.8 |
| 0.4 | 65. | 63. | 59. | 64. | 60. | 62.2 |
| 0.5 | 64. | 62. | 60. | 64. | 59. | 61.8 |
| 0.6 | 64. | 62. | 60. | 63. | 59. | 61.6 |
| 0.7 | 63. | 61. | 59. | 63. | 59. | 61.0 |
| 0.8 | 63. | 62. | 59. | 62. | 60. | 61.2 |
| 0.9 | 64. | 62. | 59. | 62. | 60. | 61.4 |
| 1.0 | 63. | 61. | 59. | 62. | 60. | 61.0 |
| 1.1 | 63. | 61. | 58. | 63. | 60. | 61.0 |
| 1.2 | 63. | 62. | 58. | 64. | 60. | 61.4 |
| 1.3 | 62. | 62. | 58. | 63. | 60. | 61.0 |
| 1.4 | 62. | 61. | 58. | 64. | 60. | 61.0 |
| 1.5 | 64. | 61. | 58. | 64. | 61. | 61.6 |
| 1.6 | 64. | 60. | 58. | 65. | 60. | 61.4 |
| 1.7 | 64. | 59. | 58. | 64. | 59. | 60.8 |
| 1.8 | 64. | 59. | 58. | 64. | 59. | 60.8 |
| 1.9 | 64. | 60. | 59. | 64. | 59. | 61.2 |
| 2.0 | 64. | 60. | 60. | 64. | 59. | 61.4 |
| 2.1 | 64. | 60. | 59. | 64. | 59. | 61.2 |
| 2.2 | 64. | 61. | 59. | 63. | 60. | 61.4 |
| 2.3 | 64. | 61. | 58. | 63. | 60. | 61.2 |
| 2.4 | 64. | 62. | 58. | 63. | 60. | 61.4 |
| 2.5 | 65. | 61. | 58. | 63. | 60. | 61.4 |
| 2.6 | 65. | 60. | 58. | 63. | 59. | 61.0 |
| 2.7 | 65. | 59. | 59. | 62. | 59. | 60.8 |
| 2.8 | 65. | 60. | 59. | 62. | 60. | 61.2 |
| 2.9 | 64. | 60. | 58. | 62. | 61. | 61.0 |
| 3.0 | 64. | 61. | 57. | 62. | 60. | 60.8 |
| 3.1 | 63. | 61. | 57. | 63. | 59. | 60.6 |
| 3.2 | 63. | 61. | 57. | 62. | 58. | 60.2 |
| 3.3 | 63. | 60. | 58. | 62. | 58. | 60.2 |
| 3.4 | 63. | 60. | 58. | 63. | 60. | 60.8 |
| 3.5 | 64. | 59. | 59. | 63. | 60. | 61.0 |
| 3.6 | 64. | 60. | 58. | 62. | 59. | 60.6 |
| 3.7 | 63. | 61. | 58. | 63. | 61. | 61.2 |
| 3.8 | 63. | 62. | 58. | 62. | 61. | 61.2 |
| 3.9 | 64. | 62. | 58. | 63. | 60. | 61.4 |
| 4.0 | 64. | 61. | 59. | 64. | 60. | 61.6 |
| 4.1 | 64. | 61. | 60. | 63. | 60. | 61.6 |
| 4.2 | 64. | 59. | 60. | 63. | 58. | 60.8 |
| 4.3 | 64. | 60. | 60. | 63. | 58. | 61.0 |
| 4.4 | 64. | 61. | 59. | 62. | 59. | 61.0 |
| 4.5 | 63. | 61. | 59. | 62. | 58. | 60.6 |
| 4.6 | 63. | 59. | 59. | 62. | 58. | 60.2 |
| 4.7 | 63. | 60. | 59. | 62. | 58. | 60.4 |
| 4.8 | 63. | 60. | 60. | 62. | 60. | 61.0 |
| 4.9 | 64. | 60. | 60. | 62. | 60. | 61.2 |
| 5.0 | 64. | 59. | 59. | 62. | 60. | 60.8 |
| 5.1 | 65. | 59. | 58. | 63. | 58. | 60.6 |
| 5.2 | 65. | 61. | 59. | 64. | 57. | 61.2 |
| 5.3 | 64. | 61. | 58. | 63. | 58. | 60.8 |
| 5.4 | 64. | 61. | 58. | 62. | 60. | 61.0 |
| 5.5 | 65. | 61. | 58. | 63. | 60. | 61.4 |
| 5.6 | 65. | 61. | 59. | 62. | 59. | 61.2 |
| 5.7 | 66. | 61. | 60. | 61. | 59. | 61.4 |
| 5.8 | 66. | 61. | 61. | 61. | 59. | 61.1 |
| 5.9 | 67. | 61. | 60. | 61. | 59. | 61.6 |
| 6.0 | 66. | 63. | 59. | 61. | 59. | 61.6 |
| 6.1 | 65. | 63. | 58. | 62. | 61. | 61.8 |
| 6.2 | 65. | 63. | 58. | 61. | 60. | 61.4 |
| 6.3 | 65. | 63. | 60. | 62. | 60. | 62.0 |
| 6.4 | 65. | 63. | 60. | 62. | 60. | 62.0 |
| 6.5 | 65. | 63. | 60. | 62. | 61. | 62.2 |
| 6.6 | 64. | 63. | 59. | 62. | 60. | 61.6 |
| 6.7 | 64. | 63. | 59. | 63. | 59. | 61.6 |
| 6.8 | 65. | 63. | 59. | 64. | 59. | 62.0 |
| 6.9 | 65. | 62. | 59. | 64. | 59. | 61.8 |
| 7.0 | 64. | 62. | 60. | 64. | 59. | 61.8 |
| 7.1 | 64. | 62. | 61. | 64. | 59. | 62.0 |
| 7.2 | 64. | 62. | 60. | 63. | 60. | 61.8 |
| 7.3 | 64. | 62. | 60. | 63. | 59. | 61.6 |
| 7.4 | 64. | 62. | 59. | 63. | 58. | 61.2 |
| 7.5 | 65. | 61. | 59. | 61. | 58. | 60.8 |
| 7.6 | 65. | 60. | 59. | 61. | 58. | 60.6 |
| 7.0 | 65. | 62. | 60. | 62. | 58. | 61.4 |
| 7.8 | 65. | 62. | 60. | 61. | 58. | 61.2 |
| 7.9 | 64. | 61. | 60. | 63. | 59. | 61.4 |
| 8.0 | 64. | 61. | 59. | 64. | 59. | 61.4 |
| 8.1 | 65. | 61. | 59. | 64. | 59. | 61.6 |
| 8.2 | 65. | 61. | 59. | 64. | 58. | 61.4 |
| 8.3 | 65. | 60. | 59. | 63. | 58. | 61.0 |
| 8.4 | 65. | 60. | 60. | 63. | 58. | 61.2 |
| 8.5 | 65. | 60. | 60. | 62. | 59. | 61.2 |
| 8.6 | 65. | 61. | 60. | 61. | 59. | 61.2 |
| 8.7 | 66. | 61. | 61. | 61. | 59. | 61.6 |
| 8.8 | 66. | 61. | 60. | 62. | 59. | 61.6 |
| 8.9 | 66. | 61. | 60. | 62. | 59. | 61.6 |
| 9.0 | 66. | 61. | 61. | 61. | 59. | 61.6 |
| 9.1 | 65. | 61. | 59. | 61. | 59. | 61.0 |
| 9.2 | 65. | 61. | 59. | 62. | 59. | 61.2 |
| 9.3 | 65. | 61. | 59. | 62. | 59. | 61.2 |
| 9.4 | 65. | 62. | 60. | 64. | 59. | 62.0 |
| 9.5 | 64. | 63. | 60. | 64. | 60. | 62.2 |
| 9.6 | 64. | 61. | 60. | 64. | 61. | 62.0 |
| 9.7 | 64. | 61. | 61. | 63. | 59. | 61.6 |
| 9.8 | 64. | 61. | 60. | 63. | 59. | 61.4 |
| 9.9 | 65. | 61. | 60. | 62. | 59. | 61.4 |
| 10.0 | 66. | 62. | 61. | 62. | 59. | 62.0 |

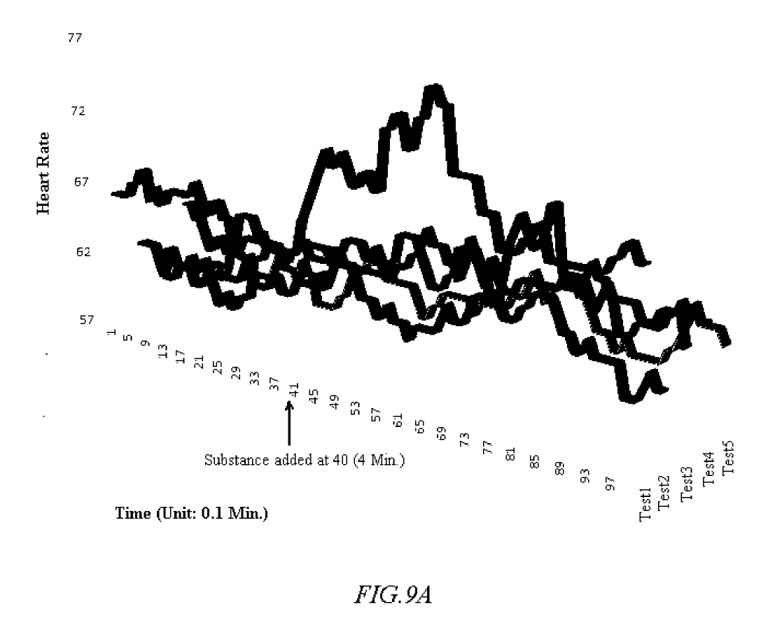

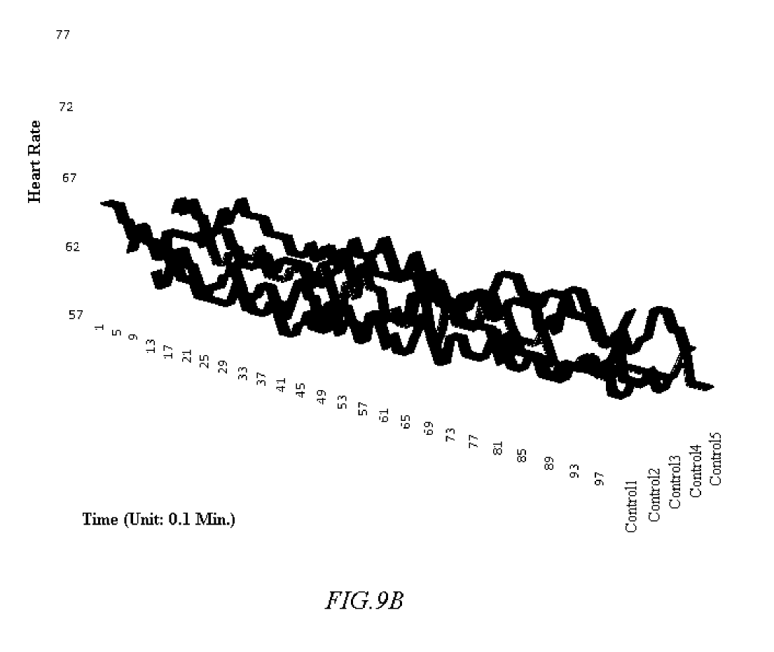

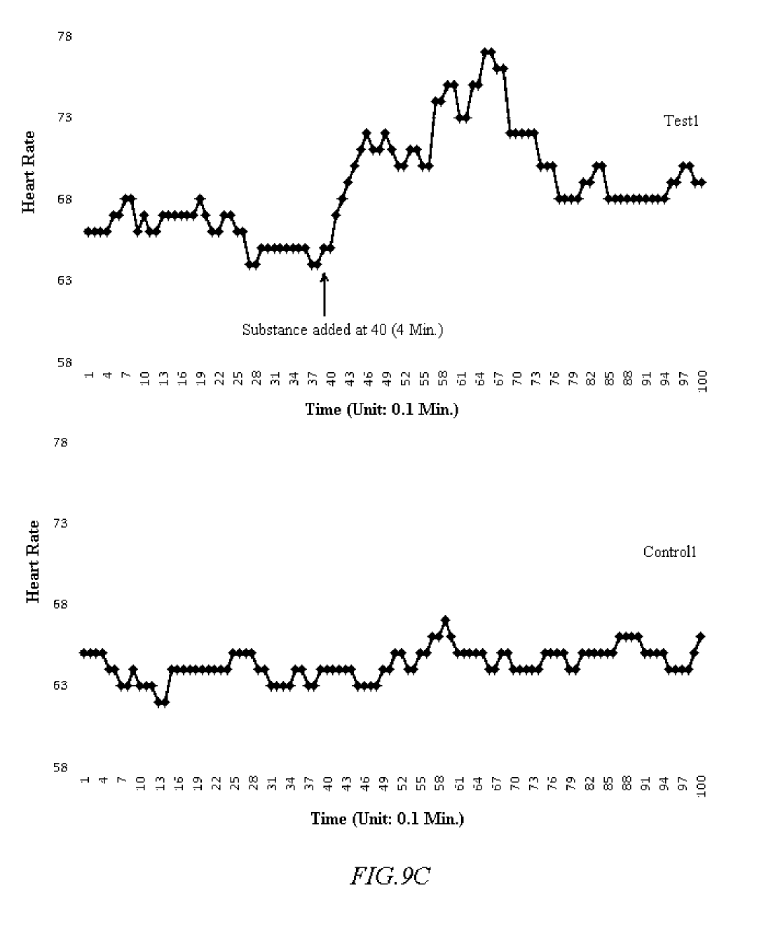

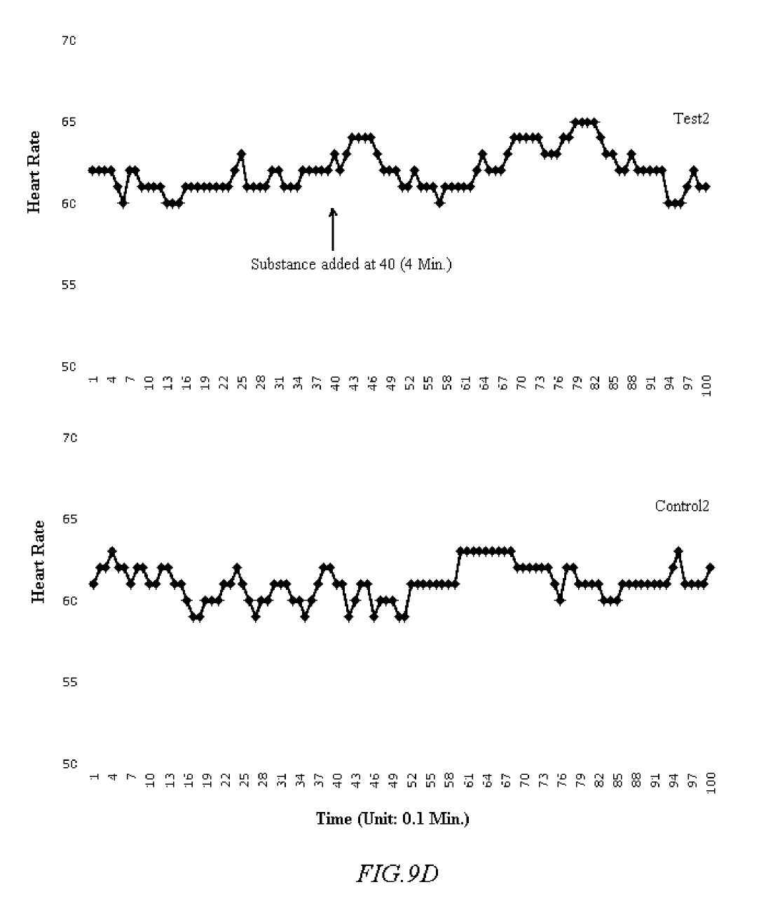

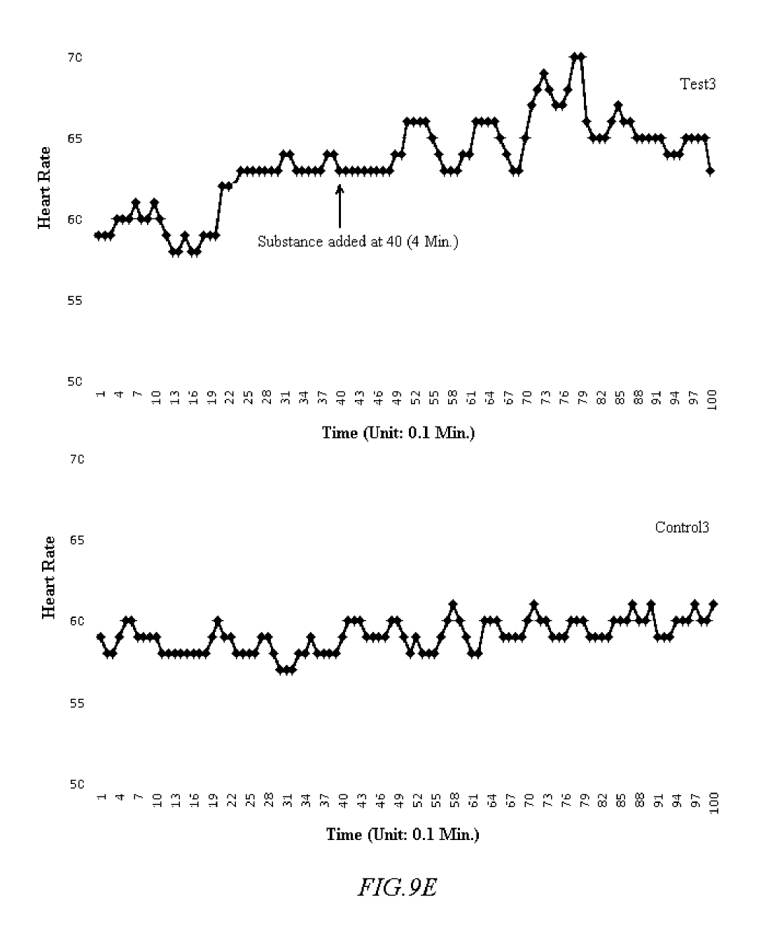

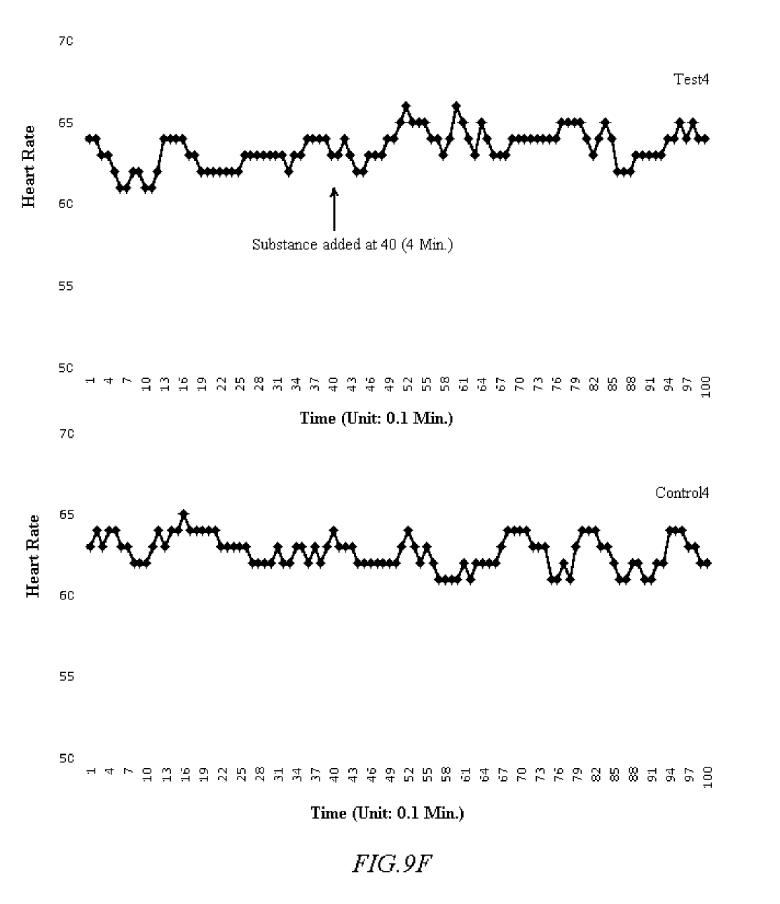

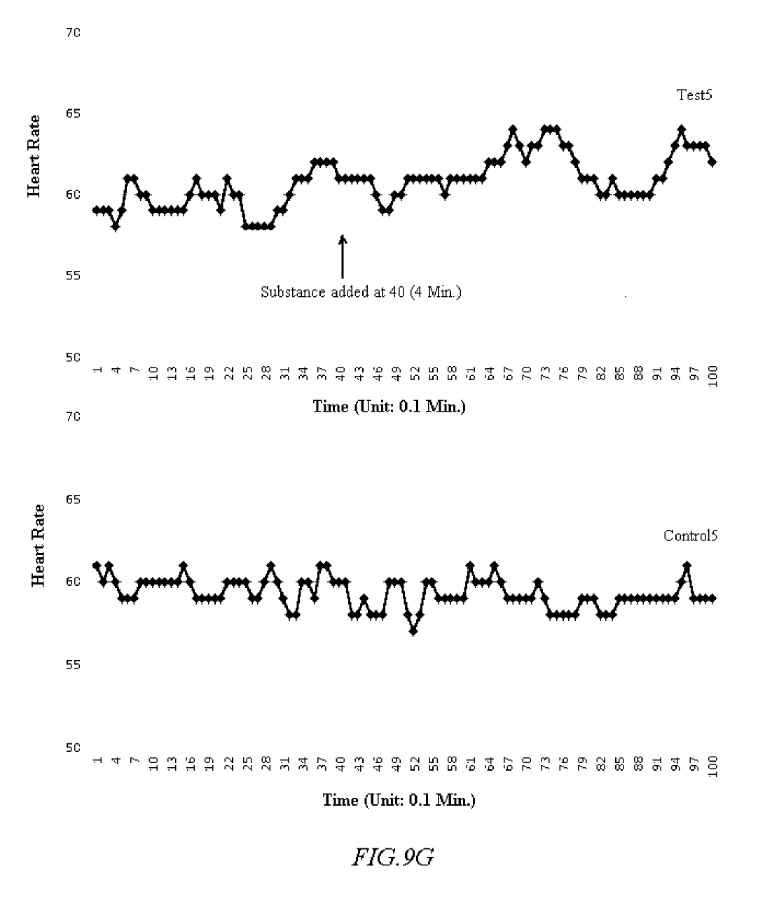

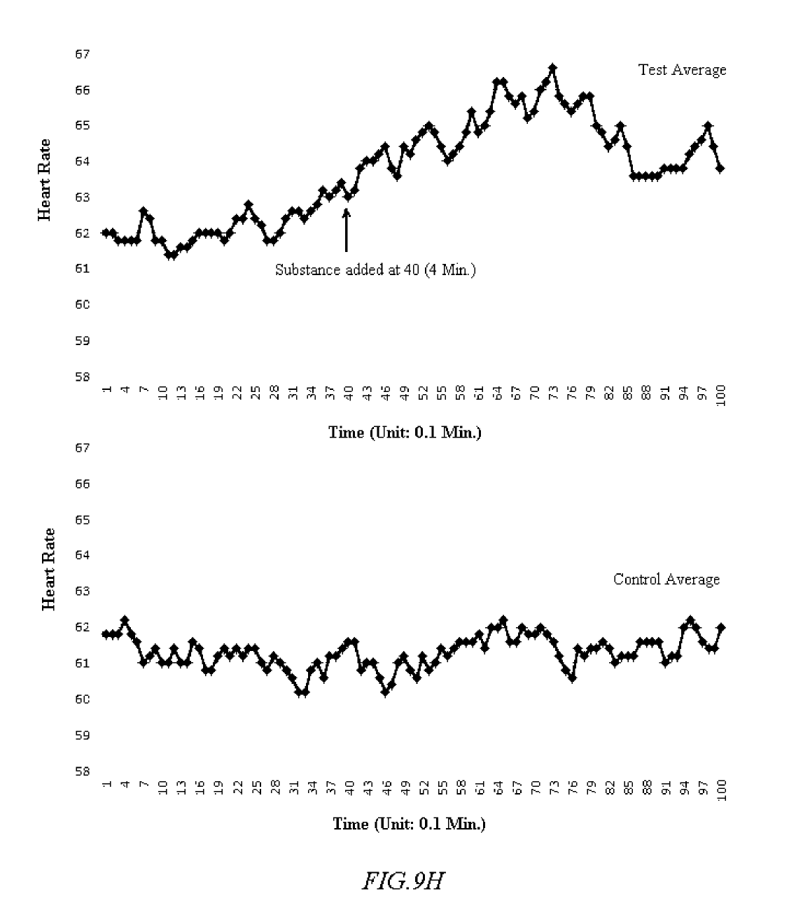

FIG. 9A is a 3D chart of the time series of heart rate in Table 4 and FIG. 9B is a 3D chart of the time series of heart rate in Table 5. FIG. 9C, FIG. 9D, FIG. 9E, FIG. 9F and FIG. 9G are respectively the time series charts of heart rate for the experimental pairs (Test1, Control1), (Test2, Control2), (Test3, Control3), (Test4, Control4) and (Test5, Control5). Further, FIG. 9H is the chart of the mean of the time series in Table 4 and Table 5.

It is clear from Tables 4 and 5 and FIG. 9A-9H, while the baselines (controls) of the heart rate of Subject B are stable, and fluctuate and drift within the ranges of five (5) points (beats), Primatene solution containing 60 mg ephedrine produced detectable non-local effect in Subject B in the form of rapidly increased heart rate for at least four (4) minutes in the range of 1-6 points (beats) or 1.5%-10% above the fluctuating ranges of the baselines.

The increase of heart rate in Subject B non-locally induced by Primatene is statistically significant because: (1) The timing of the increase in heart rate coincide with the time of adding Primatene; (2) Averaging of the time series does not cancel the signal as random noises would as shown in FIG. 9H; and (3) student's t-test comparing data points within 4 minutes (forty data points) immediately after adding Primatene (Sample1) and the data points within 4 minutes (forty data points) immediately before adding Primatene (Sample2) all shows statistically significant differences as shown in Table 6 below:

TABLE 6

| Test1 | N | Mean | St Dev | SE Mean |
|---|---|---|---|---|
| Sample1 | 40 | 71.65 | 2.6559 | 0.42 |
| Sample2 | 40 | 66 | 1.1323 | 0.179 |

Observed difference (Sample 1 − Sample 2): 5.65
Standard Deviation of Difference: 0.4565
DF: 52
95% Confidence Interval for the Difference (4.734, 6.566)
T-Value 12.3768
Population 1 ≠ Population 2: P-Value = <.00001
Population 1 > Population 2: P-Value = >.99999
Population 1 < Population 2: P-Value = <.00001

| Test2 | N | Mean | StDev | SE Mean |
|---|---|---|---|---|
| Sample 1 | 40 | 62.575 | 1.3376 | 0.211 |
| Sample 2 | 40 | 61.35 | 0.7355 | 0.116 |

Observed difference (Sample 1 − Sample 2): 1.225
Standard Deviation of Difference: 0.2414
DF: 60
95% Confidence Interval for the Difference (0.7421, 1.7079)
T-Value 5.0746
Population 1 ≠ Population 2: P-Value = <.00001
Population 1 > Population 2: P-Value = >.99999
Population 1 < Population 2: P-Value = <.00001

| Test3 | N | Mean | StDev | SE Mean |
|---|---|---|---|---|
| Sample 1 | 40 | 65.15 | 2.0946 | 0.331 |
| Sample 2 | 40 | 61.175 | 2.0492 | 0.324 |

Observed difference (Sample 1 − Sample 2): 3.975
Standard Deviation of Difference: 0.4633
DF: 77
95% Confidence Interval for the Difference (3.0524, 4.8976)
T-Value 8.5798
Population 1 ≠ Population 2: P-Value = <.00001
Population 1 > Population 2: P-Value = >.99999
Population 1 < Population 2: P-Value = <.00001

| Test4 | N | Mean | StDev | SE Mean |
|---|---|---|---|---|
| Sample 1 | 40 | 64 | 0.9608 | 0.152 |
| Sample 2 | 40 | 62.75 | 0.9541 | 0.151 |

Observed difference (Sample 1 − Sample 2): 1.25
Standard Deviation of Difference: 0.2141
DF: 77
95% Confidence Interval for the Difference (0.8237, 1.6763)
T-Value 5.8384
Population 1 ≠ Population 2: P-Value = <.00001
Population 1 > Population 2: P-Value = >.99999
Population 1 < Population 2: P-Value = <.00001

| Test5 | N | Mean | StDev | SE Mean |
|---|---|---|---|---|
| Sample 1 | 40 | 61.525 | 1.3006 | 0.206 |
| Sample 2 | 40 | 59.775 | 1.2297 | 0.194 |

Observed difference (Sample 1 − Sample 2): 1.75
Standard Deviation of Difference: 0.283
DF: 77
95% Confidence Interval for the Difference (1.1865, 2.3135)
T-Value 6.1837
Population 1 ≠ Population 2: P-Value = <.00001
Population 1 > Population 2: P-Value = >.99999
Population 1 < Population 2: P-Value = <.00001

Table 7 summarize two (2) sets of measurement data obtained on Subject C with the same procedure as that used on Subject B, each set comprising a test and a control (baseline), 10 ml solution of five (5) tablets of Primatene (containing 60 mg ephedrine) being added at the marked time of four (4) minute:

TABLE 7

| Time | Test1 | Ctrl1 | Test2 | Ctrl2 | |
|---|---|---|---|---|---|
| 0.1 | 73. | 74. | 62. | 63. | |
| 0.2 | 72. | 73. | 61. | 62. | |
| 0.3 | 73. | 74. | 61. | 62. | |
| 0.4 | 74. | 74. | 61. | 63. | |
| 0.5 | 73. | 73. | 62. | 62. | |
| 0.6 | 75. | 73. | 63. | 62. | |
| 0.7 | 74. | 73. | 62. | 64. | |
| 0.8 | 74. | 72. | 62. | 63. | |
| 0.9 | 73. | 72. | 63. | 63. | |
| 1.0 | 73. | 72. | 63. | 63. | |
| 1.1 | 72. | 72. | 64. | 63. | |
| 1.2 | 71. | 73. | 64. | 62. | |
| 1.3 | 72. | 74. | 64. | 63. | |
| 1.4 | 73. | 73. | 62. | 62. | |
| 1.5 | 74. | 72. | 62. | 62. | |
| 1.6 | 73. | 73. | 62. | 61. | |
| 1.7 | 72. | 74. | 62. | 61. | |
| 1.8 | 74. | 75. | 62. | 62. | |
| 1.9 | 73. | 76. | 63. | 64. | |
| 2.0 | 72. | 75. | 63. | 63. | |
| 2.1 | 72. | 74. | 63. | 64. | |
| 2.2 | 73. | 75. | 63. | 63. | |
| 2.3 | 74. | 74. | 63. | 61. | |
| 2.4 | 75. | 73. | 64. | 61. | |
| 2.5 | 74. | 74. | 65. | 61. | |
| 2.6 | 73. | 75. | 64. | 61. | |
| 2.7 | 73. | 74. | 63. | 59. | |
| 2.8 | 72. | 73. | 62. | 60. | |
| 2.9 | 73. | 73. | 62. | 62. | |
| 3.0 | 73. | 73. | 63. | 63. | |
| 3.1 | 74. | 73. | 63. | 63. | |
| 3.2 | 73. | 74. | 63. | 65. | |
| 3.3 | 72. | 71. | 63. | 64. | |
| 3.4 | 71. | 71. | 63. | 61. | |
| 3.5 | 70. | 71. | 64. | 62. | |
| 3.6 | 71. | 72. | 63. | 61. | |
| 3.7 | 70. | 73. | 63. | 61. | |
| 3.8 | 71. | 72. | 62. | 61. | |
| 3.9 | 72. | 72. | 62. | 61. | |
| 4.0 | 73. | 72. | 63. | 61. | (Added Primatene Here |
| | | | | | in Test1 and Test2) |
| 4.1 | 72. | 75. | 64. | 61. | |
| 4.2 | 73. | 75. | 65. | 61. | |
| 4.3 | 74. | 74. | 67. | 61. | |
| 4.4 | 75. | 75. | 66. | 62. | |
| 4.5 | 78. | 76. | 66. | 63. | |
| 4.6 | 79. | 77. | 64. | 63. | |
| 4.7 | 80. | 76. | 65. | 62. | |
| 4.8 | 79. | 75. | 65. | 62. | |
| 4.9 | 78. | 74. | 63. | 62. | |
| 5.0 | 77. | 74. | 62. | 62. | |
| 5.1 | 77. | 75. | 64. | 62. | |
| 5.2 | 76. | 74. | 65. | 65. | |
| 5.3 | 75. | 75. | 67. | 64. | |
| 5.4 | 74. | 74. | 65. | 63. | |
| 5.5 | 73. | 74. | 64. | 62. | |
| 5.6 | 74. | 75. | 63. | 62. | |
| 5.7 | 73. | 76. | 64. | 62. | |
| 5.8 | 74. | 75. | 64. | 62. | |
| 5.9 | 76. | 74. | 63. | 62. | |
| 6.0 | 77. | 75. | 63. | 63. | |
| 6.1 | 76. | 76. | 64. | 63. | |
| 6.2 | 74. | 75. | 64. | 63. | |
| 6.3 | 73. | 74. | 63. | 62. | |
| 6.4 | 74. | 74. | 63. | 62. | |
| 6.5 | 74. | 75. | 63. | 64. | |
| 6.6 | 76. | 74. | 62. | 62. | |
| 6.7 | 77. | 75. | 62. | 61. | |
| 6.8 | 76. | 73. | 62. | 60. | |
| 6.9 | 74. | 74. | 62. | 60. | |
| 7.0 | 73. | 73. | 63. | 61. | |
| 7.1 | 72. | 73. | 64. | 62. | |
| 7.2 | 73. | 72. | 64. | 62. | |
| 7.3 | 72. | 73. | 63. | 64. | |
| 7.4 | 71. | 74. | 62. | 65. | |
| 7.5 | 72. | 73. | 61. | 61. | |
| 7.6 | 71. | 75. | 62. | 61. | |
| 7.0 | 70. | 74. | 63. | 62. | |
| 7.8 | 71. | 73. | 63. | 62. | |
| 7.9 | 72. | 73. | 63. | 62. | |
| 8.0 | 73. | 73. | 63. | 62. | |
| 8.1 | 74. | 72. | 63. | 60. | |
| 8.2 | 75. | 71. | 63. | 61. | |
| 8.3 | 76. | 72. | 63. | 62. | |
| 8.4 | 75. | 73. | 61. | 62. | |
| 8.5 | 74. | 74. | 62. | 62. | |
| 8.6 | 73. | 73. | 62. | 62. | |
| 8.7 | 71. | 72. | 62. | 61. | |
| 8.8 | 72. | 74. | 62. | 61. | |
| 8.9 | 73. | 73. | 62. | 61. | |
| 9.0 | 72. | 72. | 62. | 61. | |
| 9.1 | 73. | 72. | 62. | 62. | |
| 9.2 | 74. | 73. | 63. | 61. | |
| 9.3 | 73. | 74. | 64. | 61. | |
| 9.4 | 74. | 75. | 66. | 61. | |
| 9.5 | 73. | 74. | 63. | 62. | |
| 9.6 | 72. | 73. | 62. | 63. | |
| 9.7 | 73. | 73. | 64. | 62. | |
| 9.8 | 74. | 72. | 64. | 62. | |
| 9.9 | 76. | 73. | 65. | 63. | |
| 10.0 | 78. | 73. | 65. | 63. | |

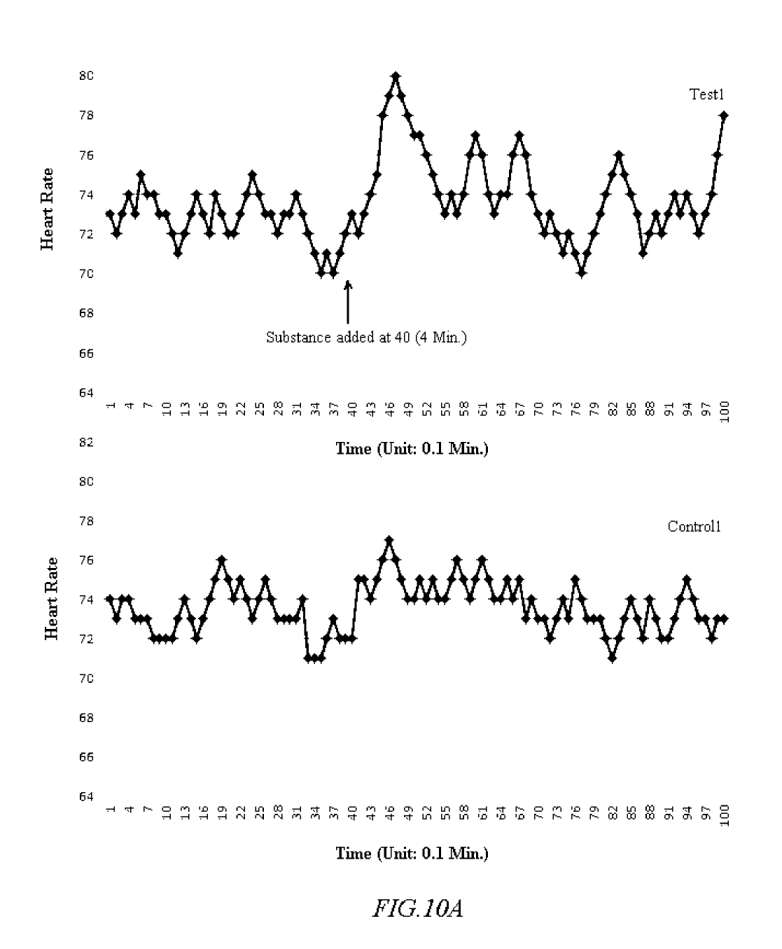

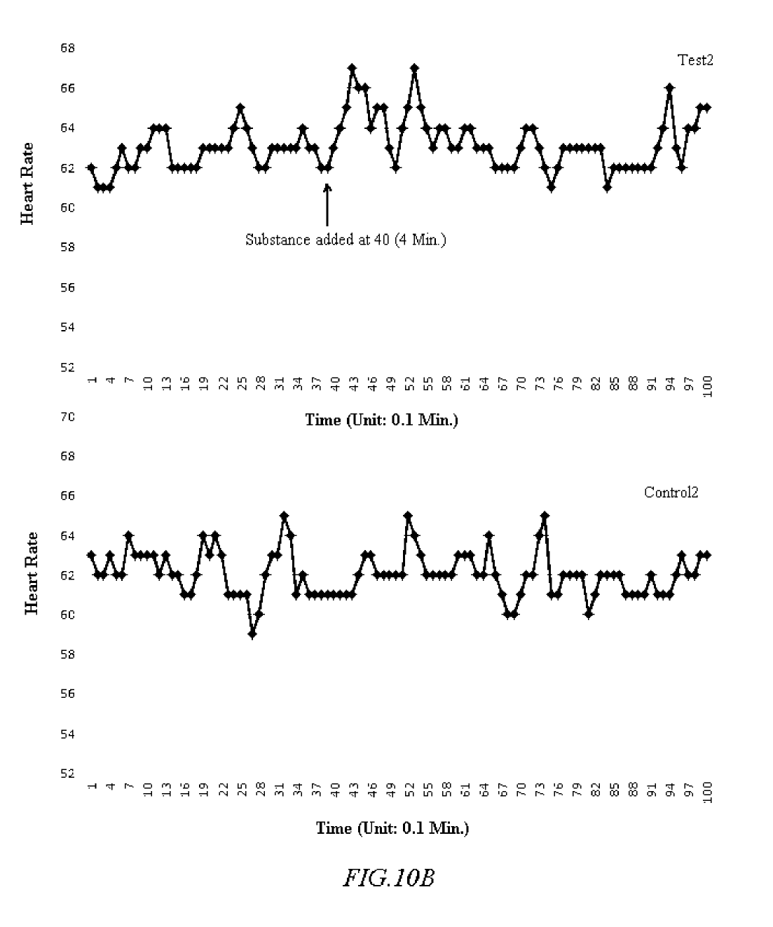

FIG. 10A and FIG. 10B are respectively the time series charts of heart rate for the experimental pairs (Test1, Control1) and (Test2, Control2) conducted on Subject C. It can been seen from Table 7 and FIG. 10A-B, while the baselines (controls) of the heart rate of Subject C are stable and fluctuate within the ranges of five (6) points (beats), Primatene solution containing 60 mg ephedrine produced detectable non-local effect in Subject C in the form of rapidly increased heart rate for at least two (2) minutes in the range of 1-4 points (beats) or 1.5%-6% above the fluctuating ranges of the baselines.

Student's t-test on data sets collected on Subject C comparing data points within 4 minutes (forty data points) immediately after adding Primatene (Sample1) and the data points within 4 minutes (forty data points) immediately before adding Primatene (Smaple2) show statistically significant differences as shown in Table 8 below:

TABLE 8

| Test1 | N | Mean | StDev | SE Mean |
|---|---|---|---|---|
| Sample 1 | 40 | 74.45 | 2.4698 | 0.391 |
| Sample 2 | 40 | 72.725 | 1.198 | 0.189 |

Observed difference (Sample 1 − Sample 2): 1.725
Standard Deviation of Difference: 0.434
DF: 56
95% Confidence Interval for the Difference (0.8556, 2.5944)
T-Value 3.9747
Population 1 ≠ Population 2: P-Value = 0.0002
Population 1 > Population 2: P-Value = 0.9999
Population 1 < Population 2: P-Value = 0.0001

| Test2 | N | Mean | StDev | SE Mean |
|---|---|---|---|---|
| Sample 1 | 40 | 63.625 | 1.3902 | 0.22 |
| Sample 2 | 40 | 62.725 | 0.9055 | 0.143 |

Observed difference (Sample 1 − Sample 2): 0.9
Standard Deviation of Difference: 0.2623
DF: 67
95% Confidence Interval for the Difference (0.3764, 1.4236)
T-Value 3.4312
Population 1 ≠ Population 2: P-Value = 0.001
Population 1 > Population 2: P-Value = 0.9995
Population 1 < Population 2: P-Value = 0.0005

Further, experiments conducted on Subject A with Primatene while exploring the modes of experimental setup showed increased heart rate in Subject A.

Therefore, the applicant concludes that the biological and/or chemical effects such as brain effects experienced by the test subjects were the consequences of quantum entanglement between quantum entities inside the biological and/or chemical systems such as the brains and those of the applied chemical substances induced by the entangling photons of the magnetic pulses or applied lights.

In light of the forgoing, the results obtained in the first set of experiments can be interpreted as the consequence of quantum entanglement induced by the photons of the magnetic pulses. Similarly, the results obtained from the second sets of experiments can be explained as quantum entanglement between the quantum entities in the water and those in the chemical substance induced by the photons of the laser light or magnetic pulses and the subsequent physical transport of the water to a biological and/or chemical system such as the brain after consumption by the test subject which, in turn, produces the observed biological and/or chemical effects such as brain effects through the entanglement of the quantum entities inside the biological and/or chemical system such as the brain with those in the consumed water.

We further conclude that the biological and/or chemical effects such as changes of heart rate in the test subjects were produced through a second quantum entanglement between quantum entities inside the biological and/or chemical systems such as the heart and associated nervous systems and those of the applied chemical substances such as Primatene, which in turn is mediated by the quantum entanglement between two parts of quantum entangled medium such as microwaved water.

While the applicant does not wish to be bound by any particular quantum entities suggested herein, it is believed that nuclear spins and/or electron spins respectively inside the substance and biological and/or chemical system such as the brain are the quantum entities responsible for mediating the non-local effect of the substance on the biological and/or chemical system such as the brain since nuclear spins and electron spins are the natural targets of interaction with the quantum-entangling members such as photons for reasons discussed below.

The applicant first chose general anaesthetics in his experiments because the said substances are among the most powerful brain-influencing substances. The applicant's expectation was that, if nuclear and/or electronic spins inside the brain are involved in brain functions such as perception as recently hypothesized by the applicant with his collaborator (Hu, H. P., & Wu, M. X. Spin-mediated consciousness theory. Medical Hypotheses 63, 633-646 (2004). Also see arXiv e-print quant-ph/0208068 (2002)), the brain would be able to sense the effect of an external anaesthetic sample through quantum entanglement between these spins inside the brain and those in the said anesthetic sample induced by the photons of the magnetic pulse or laser light by first interacting with the nuclear spins in the said anesthetic sample, thus carrying quantum information about the anesthetic molecules, and then interacting with the nuclear and/or electronic spins inside the brain. In turn, the brain will be able to sense the effect of the general anesthetic because of the resulting quantum entanglement.

Indeed, neural membranes and proteins contain vast numbers of nuclear spins such as ¹H, ¹³C, ³¹P and ¹⁵N. These nuclear spins and unpaired electron spins are natural targets of interaction with the photons of the magnetic pulse or laser light. Indeed, these spins form complex intra- and inter-molecular networks through various intra-molecular J- and dipolar couplings and both short- and long-range intermolecular dipolar couplings. Further, nuclear spins have long relaxation times after excitations (Hu, H. & Wu, M. Action potential modulation of neural spin networks suggests possible role of spin in memory and consciousness NeuroQuantology 2:309-317 (2004)). Thus, when a nematic liquid crystal is irradiated with multi-frequency pulse magnetic fields, its ¹H spins can form long-lived intra-molecular quantum coherence with entanglement for information storage (Khitrin, A. K., Ermakov, V. L. & Fung, B. M. Information storage using a cluster of dipolar-coupled spins. Chem. Phys. Lett. 360, 161-166 (2002)). Also, long-lived (˜0.05 ms) entanglement of two macroscopic electron spin ensembles in room temperature has also been achieved (Julsgaard, B., Kozhekin, A. & Polzik, E. S. Experimentally long-lived entanglement of two macroscopic objects. Nature 413, 400-403 (2001)). Conceptually, spin is a fundamental quantum process with intrinsic connection to the structure of space-time and was shown to be responsible for the quantum effects in both Hestenes and Bohmian quantum mechanics (Hu, H. & Wu, M. Spin as primordial self-referential process driving quantum mechanics, spacetime dynamics and consciousness. NeuroQuantology 2:41-49 (2004)). Thus, the applicant has recently suggested with his collaborator that these spins could be involved in brain functions at a more fundamental level (see Hu, H. P., & Wu, M. X. Spin-mediated consciousness theory. Medical Hypotheses 63, 633-646 (2004). Also see arXiv e-print quant-ph/0208068 (2002)).

The applicant would like to point out that although some of the indicators used to measure the biological and/or chemical effects such as brain effects in the experiments carried out by him were qualitative and subjective without using an objective and quantitative detecting device, they reflect the first-person experiences of the qualities, intensities and durations of these biological and/or chemical effects such as the brain effects by the test subjects since their brains were directly used as experimental probes. Further, these effects were completely reproducible under blind experimental settings so that possible placebo effects were excluded.

My invention and discovery make it clear that (1) biologically and/or chemically meaningful information can be transmitted from one system or location to the other through quantum entanglement; (2) quantum entanglement can be used to deliver the therapeutic effects of many drugs to biological systems such as human bodies without ever physically administrating the said drugs to the said systems; (3) quantum entanglement alone can be used for communications of both quantum and classical information; (4) many substances of nutritional and even recreational values can be repeatedly administrated to the human body through the said technologies; (5) it can be used for instantaneous communications with humans sent to the outer space; and (6) quantum entanglement can also be used to entangle two or more human minds for legitimate purposes.

Besides the various experiments described above, the following examples will further illustrate specific embodiments of the present invention, with the end use applications for which each is particularly preferred.

### Example 1

The small glassware was filled with 20 ml CHCl3 or the medication containing morphine. It was found by simply disposing the small glassware filled with one of the said substance next to the test subject's forehead for several hours as illustrated in FIG. 1B, weak but noticeably brain effect was produced.

### Example 2

It was found that drinking water exposed to photons of a quantum entanglement generating source such as magnetic coil, laser or microwave in the presence of a general anesthetic such as CHCl3, diethyl ether, isoflorance or tribromoethanol, or a medication containing morphine, fentanyl or oxycodone in various manners disclosed herein suppressed pain in the test subjects.

### Example 3

It was further found that drinking one part of the quantum entangled water while exposing the other part of the same water photons of the magnetic coil or laser in the presence of a general anesthetic such as CHCl3 or diethyl ether, or a medication containing morphine in the manners disclosed herein also suppressed pain in the test subjects regardless of the distance between the locations of consumption and exposure.

It will be evident from the above that there are other embodiments which are clearly within the scope and spirit of the present invention, although they were not expressly set forth above. Therefore, the above disclosure is exemplary only, and the actual scope of my invention is to be determined by the claims.

What is claimed is:

1. A method of producing and detecting a quantum entanglement between a first target and a second target, and a first non-local effect of said second target on said first target through said quantum entanglement and/or a second non-local effect of said first target on said second target through said quantum entanglement, which comprises the steps of:
selecting said first target which comprises a first substance, mixture of substances, physical system, chemical system or biological system;
selecting said second target which comprises a second substance, mixture of substances, physical system, chemical system or biological system;
providing quantum entanglement generating source which emits a plurality of quantum entanglement generating members when said source operates;
providing a detecting mean which detects said quantum entanglement, said first non-local effect and/or said second non-local effect when said mechanism operates;
disposing said first target between said source and said second target or said second target between said source and said first target;
driving said source to emit said generating members which interact with said first target and said second target; and
detecting said quantum entanglement, said first non-local effect and/or said second non-local effect;
whereby said quantum entanglement, and said first non-local effect through said quantum entanglement and/or said second non-local effect through said quantum entanglement are generated and detected, said non-local effects being physical or chemical, or biological in a human or animal.

2. A method as in claim 1 wherein said first target comprises a first chemical substance, water based medium, human or animal; said second target comprises a second chemical substance, water based medium, human or animal; and said source comprises a photon or magnetic pulse generator which emits a plurality of photons or magnetic pulses as quantum entanglement generating members when said generator operates.

3. A method as in claim 2 wherein said first target comprises an anesthetic, therapeutic, recreational, communicational, brain or heart stimulating, performance or health enhancing, or disease preventing substance; said second target comprises said second human or animal; said source comprises a magnetic coil connected to a driving mechanism, laser or microwave device; and said detecting mean comprises a first-person experience and report of said experience and/or a detecting device.

4. A method as in claim 3 wherein said first target comprises chloroform, isoflurance, dymethyl ether, ethanol, tribromoethanol, morphine sulfate, fentanyl, nicotine, caffeine or ephedrine; and said detecting device comprising a heart rate monitor, blood pressure monitor, EEG machine or MEG machine.

5. A method of producing and detecting a quantum entanglement between a first target and a second target, and a first non-local effect of said second target on said first target through said quantum entanglement and/or a second non-local effect of said first target on said second target through said quantum entanglement, which comprises the steps of:
selecting said first target which comprises a first substance, mixture of substances, physical system, chemical system or biological system;
selecting said second target which comprises a second substance, mixture of substances, physical system, chemical system or biological system;
providing a first medium at said first location and a second medium at said second location, said first medium being quantum-entangled with said second medium;
providing a detecting mean for detecting said quantum entanglement, said first non-local effect and/or said second non-local effect when said mechanism operates;
causing said first target to interact with said first medium through a first contact or radiation from a first source;
causing said second target to interact with said second medium through a second contact or radiation from a second source; and
detecting said quantum entanglement, said first non-local effect and/or said second non-local effect;
whereby said quantum entanglement between said first target and said second target, and said first non-local effect through said quantum entanglements and/or said second non-local effect through said quantum entanglements are generated and detected, said non-local effects being physical or chemical, or biological in a human or animal.

6. A method as in claim 5 wherein said first target comprises a first chemical substance, human or animal; said second target comprises a second chemical substance, human or animal; and said first and second media comprise a water-based medium.

7. A method as in claim 6 wherein said first target comprises an anesthetic, therapeutic, recreational, communicational, brain or heart stimulating, performance or health enhancing or disease preventing substance; said second target comprises said second human or animal; said first and second sources comprise a magnetic coil connected to a driving mechanism, laser or microwave device; and said detecting mean comprises a first-person experience and report of said experience and/or a detecting device.

8. A method as in claim 7 wherein said first target comprises chloroform, isoflurance, dymethyl ether, ethanol, tribromoethanol, morphine sulfate, fentanyl, nicotine, caffeine or ephedrine; said first contact comprises mixing said first target with said first medium; said second contact comprises delivering orally or intravenously said second medium to said second human or animal; and said detecting device comprising a heart rate monitor, blood pressure monitor, EEG machine or MEG machine.

9. A method as in claim 5 for sending an encoded message from said first or second location and receiving said encoded message at said second or first location further comprise the steps of:
encoding said message to be sent through said second or first non-local effect to said second or first location; and
decoding said message received through said second or first non-local effect at said second or first location;
whereby said encoded message from said first or second location to said second or first location is sent and received.

10. A method as in claim 9 wherein said first target comprises a first chemical substance, human or animal; said second target comprises a second chemical substance, human or animal; and said first and second media comprise a water-based medium.

11. A method as in claim 10 wherein said first target comprises an anesthetic, recreational, communicational, or brain or heart stimulating substance; said second target comprises said second human or animal; said first and second sources comprise a magnetic coil connected to a driving mechanism, laser or microwave device; and said detecting mean comprises a first-person experience and report of said experience and/or a detecting device.

12. A method as in claim 11 wherein said first target comprises chloroform, isoflurance, dymethyl ether, ethanol, tribromoethanol, morphine sulfate, fentanyl, nicotine, caffeine or ephedrine; said first contact comprises mixing said first target with said first medium; said second contact comprises delivering orally or intravenously said second medium to said second human or animal; and said detecting device comprising a heart rate monitor, blood pressure monitor, EEG machine or MEG machine.

13. An apparatus for producing and detecting a second quantum entanglement between a first target at a first location and a second target at a second location, and a first non-local effect of said second target on said first target and/or a second non-local effect of said first target on said second target, which comprises:
a first medium in a first container at said first location;
a first mean for causing said first medium to interact with said first target;
a second medium in a second container at said second location, said second medium being in a first quantum entanglement with said first medium;
a second mean for causing said second medium to interact with said second target; and
a third mean for detecting said second quantum entanglement, said first non-local effect and/or said second non-local effect;
such that when said first medium interacts with said first target and said second medium interacts with said second target, said interactions generate said second quantum entanglement through said first quantum entanglement, and said first non-local effect through said quantum entanglements and/or said second non-local effect through said quantum entanglements.

14. An apparatus as in claim 13 wherein said first and second media comprise a water-based medium; said first target comprises a first chemical substance, human or animal; said second target comprises a second chemical substance, human or animal; said first mean comprises a mean for contacting said first medium with said first target or irradiating said first medium and said first target; and said second mean comprises a mean for contacting said second medium with said second target or irradiating said second medium and said first target.

15. An apparatus as in claim 14 wherein said first target comprises an anesthetic, therapeutic, recreational, communicational, brain or heart stimulating, performance or health enhancing or disease preventing substance; said second target comprises said second human or animal; said first and second means for irradiations comprise a magnetic coil connected to a driving mechanism, laser or microwave device; and said third mean comprises a first-person experience and report of said experience and/or a detecting device.

16. An apparatus as in claim 15 wherein said first target comprises chloroform, isoflurance, dymethyl ether, ethanol, tribromoethanol, morphine sulfate, fentanyl, nicotine, caffeine or ephedrine; said first contact comprises mixing said first target with said first medium; said second contact comprises delivering orally or intravenously said second medium to said second human or animal; and said detecting device comprising a heart rate monitor, blood pressure monitor, EEG machine or MEG machine.

17. An apparatus as in claim 13 for sending an encoded message from said first or second location and receiving said encoded message at said second or first location further comprise:
a fourth mean for encoding said message to be sent through said second or first non-local effect to said second or first location; and
a fifth mean for decoding said message received through said second or first non-local effect at said second or first location;
such that said encoded message from said first or second location to said second or first location is sent and received.

18. An apparatus as in claim 17 wherein said first and second media comprise a water-based medium; said first target comprises a first chemical substance, human or animal; said second target comprises a second chemical substance, human or animal; said first mean comprises a mean for contacting said first medium with said first target or irradiating said first medium and said first target; and said second mean comprises a mean for contacting said second medium with said second target or irradiating said second medium and said first target.

19. An apparatus as in claim 18 wherein said first target comprises an anesthetic, recreational, communicational, or brain or heart stimulating substance; said second target comprises said second human or animal; said first and second means for irradiations comprise a magnetic coil connected to a driving mechanism, laser or microwave device; and said third mean comprises a first-person experience and report of said experience and/or a detecting device.

20. An apparatus as in claim 19 wherein said first target comprises chloroform, isoflurance, dymethyl ether, ethanol, tribromoethanol, morphine sulfate, fentanyl, nicotine, caffeine or ephedrine; said first contact comprises mixing said first target with said first medium; said second contact comprises delivering orally or intravenously said second medium to said second human or animal; and said detecting device comprising a heart rate monitor, blood pressure monitor, EEG machine or MEG machine.
